SEQUENCE LISTING (16 sequences)

SEQ ID NO: 1 - DNA, 1617 bp, Human herpesvirus type 6B
  CDS (67)..(1617)
ttcctcccgcggatacgcattttcgcaagactaagacggagaacggcgaccgcaacgctaagcgctatgagaccgccgagatgttctgcgccgatactcgtgtgcgcgatttccatggcgacagccctgtcgaatgctacggtctaccgagatgcgggaaccgtggaatccacaccgccgccggacgacgaagacaactataccgcaaaatactatgacgacagcatttattttaacatttacgacggtacaaatcctacgcccagaagacgaacgctacccgaaatcatctctaaattttctacctccgaaatgtcacgtctaggaggccttaaagtttttgtccccgtagattataccccaactactaccttagaagatatagaagacctcctgaactacgccatttgcgatgataacagctgcggttgtctcatagagacagaggcacgcatcatgttcggagatatcataatctgcgttcctctctcggccgaaagcagaggagtgagaaatttgaaaaacagattaatgcctatgggtctatcacagattctctctagcggcctcggccttcacttctctctgctatatggcgctttcgggagcaactataatagtttggcctatatggagcgcctgaaaccactaaccgcgatgacggcgatagctttttgtccaatgactagtaaattggagctacgccaaaactacagactggaaaaggctcgatgcgaactgatcgtcaatatcgaactcttgaagatacaaaaccatggcggacagactatcaaaacgttaacctcgtttgcgatcgttcgaaaagacaacgacgggcaagattgggaaacctgcacgcgcttcgcgtccgttagcatagaggacatattgaagtctaaacctgccgccaatggcacatgttgtcctccccgagacgtgcaccatgatcgtcctacgttacaatcttctaactcatggactcggactgagtatttcgaaccgtggcaggacgtcgttgacgcatacgtccctataaacgataatcattgtccaaacgatagctacgtcgtatttcaaaccctgcaaggtcacgaatggtgttcacgtttgaataagaacgacacgaagaattatttatcttccgtactagcgtttaaaaatgcactgtacgaaactgaggaactgatggaaaccatcggaatgcgacttgcctcacaaatattgtcactggtcggacagcgaggaacatctatacgcaacatagatccggccatcgtaagcgcattgtggcactctttgccagaaaaactgacaacgactaatattaaatacgatatagccagtccgacccatatgagtcccgcattatgcaccatctttgtacagaccggaacttcaaaacaacgtttcagaaatgcgggactactgatggttaacaacatcttcactgtccaagcgagatatagtaaacaaaatatgttcgaaaaaaaaatatatggatatgaacacctcggacaagctttatgcgagggtgggcacgtattttacaacccgagagatgtatactttcagaacatcaaaatggcagctacagaacctactgtcgtgagaacctga

SEQ ID NO: 2 - protein, 516 aa, Human herpesvirus type 6B
MRPPRCSAPILVCAISMATALSNATVYRDAGTVESTPPPDDEDNYTAKYYDDSIYFNIYDGTNPTPRRRTLPEIISKFSTSEMSRLGGLKVFVPVDYTPTTTLEDIEDLLNYAICDDNSCGCLIETEARIMFGDIIICVPLSAESRGVRNLKNRLMPMGLSQILSSGLGLHFSLLYGAFGSNYNSLAYMERLKPLTAMTAIAFCPMTSKLELRQNYRLEKARCELIVNIELLKIQNHGGQTIKTLTSFAIVRKDNDGQDWETCTRFASVSIEDILKSKPAANGTCCPPRDVHHDRPTLQSSNSWTRTEYFEPWQDVVDAYVPINDNHCPNDSYVVFQTLQGHEWCSRLNKNDTKNYLSSVLAFKNALYETEELMETIGMRLASQILSLVGQRGTSIRNIDPAIVSALWHSLPEKLTTTNIKYDIASPTHMSPALCTIFVQTGTSKQRFRNAGLLMVNNIFTVQARYSKQNMFEKKIYGYEHLGQALCEGGHVFYNPRDVYFQNIKMAATEPTVVRT

SEQ ID NO: 3 - DNA, 549 bp, Human herpesvirus type 6B
  CDS (1)..(549)
atgcatttcgtggctgtgtatattttaacacatttccatgcatatcctggagtggctgcgctaccgttttttagcacgttaccgaagattacttcgtgctgtgatcactatgtcgttcttaattcactgagttctgtatcttcctcgactccgacctgcctagacggtgaaattctgtttcaaaatgcaggccagaaattttgtcgacctttcacggacaatagaacaattgtatatactatgcaagaccaagtccaaagaccctggtccgtaacatggatggatttcaatctagtcattagtgactacgggagagctgtgatagaaaatttaaccgaaagtgctatgtcagcacacaaaaacgggccaagatatctgcagatggaaacgtttatatcggatttattccggtatgaatgtcatcgagataatcgttatgttttagaaaaaaaactacaaatgttttatccgactactcatatgaacgaattacttttttatccctcagaccctacgttgccctcaccttatggcaacggacattattaa

SEQ ID NO: 4 - protein, 182 aa, Human herpesvirus type 6B
MHFVAVYILTHFHAYPGVAALPFFSTLPKITSCCDHYVVLNSLSSVSSSTPTCLDGEILFQNAGQKFCRPFTDNRTIVYTMQDQVQRPWSVTWMDFNLVISDYGRAVIENLTESAMSAHKNGPRYLQMETFISDLFRYECHRDNRYVLEKKLQMFYPTTHMNELLFYPSDPTLPSPYGNGHY

SEQ ID NO: 5 - DNA, 2085 bp, Human herpesvirus type 6B
  CDS (1)..(2085)
atgctcttccgactctgggtctttgttctgttgactccctgttacagttggagaccgtggaccatatcgaacgagagccattgtaaaaatggaaactctgaaaatccaattgttcgcccgggctttataacttttaacttttatacaaaaaacgacactcggatatatcaagtccctaaatgcttactcggatccgatatcacgtaccatctgtttgatgccatcaacacgacagaatcgttaaccaattatgaaaaacgcgttacacgtttctatgagccgccaatgaacgatattttaagactttctaccgtacctgcggtcaagcaatttaacctggatcactctatccaaccgcagattgtctattccttaaacctgtacccttcacacggaatttattacatcagggttgtggaagtccgacagatgcaatacgacaacgtttcctgtaagctgcctaattctctcaacgaactgatctttccagtccaagttagatgcgctaaaattacacgctatgcgggcgaaaacatctatacccatttctttactccggactttatgatactgtacatccaaaatcccgcaggagatctgactatgatgtatggaaataccaccgacataaactttaaagccccttataggaaaagttcattcatattcaaacagacattgacagacgatctactattgatagtcgaaaaagacgtagtcgatgaagaataccgtttcatatcagatgcgacattcgtagacgaaacgttggatgacgtagatgaagtagaagctctactactcaaatttaataacctaggaatccaaaccctattaaggggagactgtaaaaaacccgactatgccggcataccgcagatgatgtttctttacggtatcgtacatttctcatatagcacaaaaaacacaggaccgatgcccgtgttaagagtgttaaagacacacgaaaatctcttgtccatcgactcatttgtcaaccgatgtgtgaacgtctcggaaggtacgatacaatacccaaaaatgaaggaatttttaaaatacgagccctcggactatagctacatcaccaaaaacaaatccattcccgtatctacgctgctcacgtacttagcgacagcgtacgaaaccaatgtaacgatttccaggtacaagtggtctgacattgccaacactctacaaaagatctatgaaaaacacatgttttttacgaatctgacattttccgatagggaaactctattcatgctagccgaaatagcgaatttcatccctgccgatgaacgcatgcagaggcacatgcaactactaattggaaacctgtgtaaccccgtagaaatagtttcatgggcccacatgcttacagctgacaaggcaccgaatctagaaaatatttattcgccttgtgcctctcccgtacgcagagatgtaacaaattcttttgtaaaaacagttctcacgtacgcttccctcgaccgttatcgatcagacatgatggagatgctatccgtatacagaccgccagatatggcgagagtagcggccattcagtgtctctctccaagtgagccagcagcttctctgcctctgccgaatgtgacatttgtaatttctccctcttatgtgattaaaggagtgagtttaacaattacaacgacaatcgtggctacgagtataataatcacagccatacctctcaattctacctgcgtttcaacaaattataaatatgcaggacaagatctgcttgtgctacgaaacatatcatctcaaacatgcgagttttgtcagagcgtagtcatggaatatgatgatatcgacggtcccttacaatacatctacataaaaaacatagacgaactaaaaacactgaccgatcccaacaacaatttacttgttcccaacaccaggacgcactaccttttgttagccaaaaacggctctgtttttgagatgtctgaagtcggaatcgatatagaccaagtatctatcatattggttatcatttatgttctgatcgcaataattgctttattcggcttatatagactcatcagattgtgttga

SEQ ID NO: 6 - protein, 694 aa, Human herpesvirus type 6B
MLFRLWVFVLLTPCYSWRPWTISNESHCKNGNSENPIVRPGFITFNFYTKNDTRIYQVPKCLLGSDITYHLFDAINTTESLTNYEKRVTRFYEPPMNDILRLSTVPAVKQFNLDHSIQPQIVYSLNLYPSHGIYYIRVVEVRQMQYDNVSCKLPNSLNELIFPVQVRCAKITRYAGENIYTHFFTPDFMILYIQNPAGDLTMMYGNTTDINFKAPYRKSSFIFKQTLTDDLLLIVEKDVVDEEYRFISDATFVDETLDDVDEVEALLLKFNNLGIQTLLRGDCKKPDYAGIPQMMFLYGIVHFSYSTKNTGPMPVLRVLKTHENLLSIDSFVNRCVNVSEGTIQYPKMKEFLKYEPSDYSYITKNKSIPVSTLLTYLATAYETNVTISRYKWSDIANTLQKIYEKHMFFTNLTFSDRETLFMLAEIANFIPADERMQRHMQLLIGNLCNPVEIVSWAHMLTADKAPNLENIYSPCASPVRRDVTNSFVKTVLTYASLDRYRSDMMEMLSVYRPPDMARVAAIQCLSPSEPAASLPLPNVTFVISPSYVIKGVSLTITTTIVATSIIITAIPLNSTCVSTNYKYAGQDLLVLRNISSQTCEFCQSVVMEYDDIDGPLQYIYIKNIDELKTLTDPNNNLLVPNTRTHYLLLAKNGSVFEMSEVGIDIDQVSIILVIIYVLIAIIALFGLYRLIRLC

SEQ ID NO: 7 - DNA, 753 bp, Human herpesvirus type 6B
  CDS (1)..(753)
atggaacttttactatttgtaatgtcactcatattactaaccttctcgaaagcgatgcctcttttcgaccacaattctttctattttgaaaaacttgacgactgtatcgcagctgtaataaattgcacgagatccgaagtacctctattactagaaccaatctaccaagctccggtatataatgaagacgttatgtcaatactgctaaaacccccaacaaaaaaaaagccttttagccgtataatggtaaccaatgaattcctcagcgacttcttacttctgcaggataacccagagcaactacgcacattgtttgcgctgataggggacccagaatctcgggacaattggctaaactttttcaatggcttccagacatgttcgccttccgtcggaataacaacctgcatcagcgataactgtagaaaatatttgcctgaaagaattacgtacgtcaataacttttttgttgataacattgcaggtctcgagtttaacatttcagaaaacacagacagtttttacagcaacattggttttttattatacttggagaatcctgctacaggcatcacaaaaattatcaggttcccttttaactctttgactctctttgatacgattttgaattgtttaaagtatttccacttgaaaaccggagtagaattcgacctgctaaaacagatggaagcctacaattctaaactacctttccgaagttcccgccctacgattctgattagaaacacataa

SEQ ID NO: 8 - protein, 250 aa, Human herpesvirus type 6B
MELLLFVMSLILLTFSKAMPLFDHNSFYFEKLDDCIAAVINCTRSEVPLLLEPIYQAPVYNEDVMSILLKPPTKKKPFSRIMVTNEFLSDFLLLQDNPEQLRTLFALIGDPESRDNWLNFFNGFQTCSPSVGITTCISDNCRKYLPERITYVNNFFVDNIAGLEFNISENTDSFYSNIGFLLYLENPATGITKIIRFPFNSLTLFDTILNCLKYFHLKTGVEFDLLKQMEAYNSKLPFRSSRPTILIRNT

SEQ ID NO: 9 - DNA, 622 bp, Artificial sequence
  Light chain of KH-1 antibody
ctaatacgactcactatagggcaagcagtggtatcaacgcagagtacatggggacttatgagaatagcagtaattagcnnnggaccaaaattcaaagacaaaatggattttcaggtgcagattttcagcttcctgctaatcagtgcctcagtcatactgtccagaggacaaattgttctcacccagtctccagcaatcatgtctgcatctccaggggagaaggtcaccatgacctgcagtgccagctcaagtataagttacatgcactggtaccagcagaagccaggcacctcccccaaaagatggatatatgacacatccaaactggcttctggagtccctgctcgtttcagtggcagtgggtctgggacctcttattctctcacaatcagcaacatggagtctgaagatgctgccacttattactgccatcagcggagtcgttaccacacgttcggaggggggaccaggctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtg

SEQ ID NO: 10 - protein, 207 aa, Artificial sequence
  misc_feature (27)..(27): The ′Xaa′ at location 27 stands for Lys, Asn, Arg, Ser, Thr, Ile, Met, Glu, Asp, Gly, Ala, Val, Gln, His, Pro, Leu, Tyr, Trp, Cys, or Phe.
LIRLTIGQAVVSTQSTWGLMRIAVISXGPKFKDKMDFQVQIFSFLLISASVILSRGQIVLTQSPAIMSASPGEKVTMTCSASSSISYMHWYQQKPGTSPKRWIYDTSKLASGVPARFSGSGSGTSYSLTISNMESEDAATYYCHQRSRYHTFGGGTRLEIKRADAAPTVSIFPPSSEQLTSGGASVVCFLNNFYPKDINVKWKIDGS

SEQ ID NO: 11 - DNA, 619 bp, Artificial sequence
  Heavy chain of KH-1 antibody
ctaatacgactcactatagggcaagcagtggtatcaacgcagagtacatggggatcaacatatgtccaatgtcctctccacagtccctgaagacactgactataactatgggatggacctggatctttattttaatcctgtcagtaactacaggtgtccactctgaggtccagctgcagcagtctggacctgagctggagaagcctggcgcttcagtgaagatctcctgcaaggcttctggttactcattcactggctacaacatgaactgggtgaagcagagcaatggaaagagacttgagtggattggaaatattgatccttactatggtggtgctagctacaaccagaagttcaagggcaaggccacattgactgtagacaaatcctccaccacagcctacatgcagctccagagcctgacatctgaggactctgcagtctattactgtgcaagaggggggtatggtaggtacttcgatgtctggggcgcagggaccgcggtcaccgtctcctcagccaaaacaacacccccatcagtctatccactggcccctgggtgtggagatacaactggttcctccgtgactctgggatgcctggtcaagggttatttccc

SEQ ID NO: 12 - protein, 205 aa, Artificial sequence
  Synthetic Construct
NTTHYRASSGINAEYMGINICPMSSPQSLKTLTITMGWTWIFILILSVTTGVHSEVQLQQSGPELEKPGASVKISCKASGYSFTGYNMNWVKQSNGKRLEWIGNIDPYYGGASYNQKFKGKATLTVDKSSTTAYMQLQSLTSEDSAVYYCARGGYGRYFDVWGAGTAVTVSSAKTTPPSVYPLAPGCGDTTGSSVTLGCLVKGYF

SEQ ID NO: 13 - protein, 13 aa, Human herpesvirus type 6A
QALCQGGHVFYNP

SEQ ID NO: 14 - protein, 13 aa, Artificial sequence
  HHV-6B E488Q mutant sequence
QALCQGGHVFYNP

SEQ ID NO: 15 - protein, 13 aa, Artificial sequence
  HHV-6B C487W E488Q mutant sequence
QALWQGGHVFYNP

SEQ ID NO: 16 - protein, 13 aa, Artificial sequence
  HHV-6B E488Q G489V mutant sequence
QALCQVGHVFYNP

(19) United States
(12) Patent Application Publication
(10) Pub. No.: US 2014/0093526 A1
(43) Pub. Date: Apr. 3, 2014

(54) Preparation of Neutralizing Antibody to Human Herpesvirus 6 Glycoprotein Q1 and Analysis Thereof

(71) Applicants: Yasuko Mori, Osaka (JP); Koichi Yamanishi, Osaka (JP); Michiaki Takahashi, Osaka (JP)

(72) Inventors: Yasuko Mori, Osaka (JP); Koichi Yamanishi, Osaka (JP); Michiaki Takahashi, Osaka (JP)

(73) Assignees: The Research Foundation for Microbial Diseases of Osaka University, Osaka (JP); National Institute of Biomedical Innovation, Ibaraki-shi, Osaka (JP)

(21) Appl. No.: 13/883,522

(22) PCT Filed: Apr. 15, 2011
(86) PCT No.: PCT/JP2011/002248

§ 371 Date: Dec. 18, 2013

(30) Foreign Application Priority Data
Nov. 5, 2010 (JP) 2010-249219

(51) Int. Cl. C07K 14/005; C07K 16/08; C07K 7/08

(52) U.S. Cl. CPC C07K 14/005; C07K 7/08; C07K 16/085
USPC 424/186.1; 530/327; 530/387.9; 530/350; 435/5

(57) ABSTRACT

The present invention addresses the problem of providing a vaccine which as yet has not been provided for the disease HHV-6B, which is the cause of exanthema subitum in infants, and the problem of providing an effective screening method for other therapeutic drugs. The above-mentioned problems are solved by providing an epitope specific to HHV-6B, of the amino acid sequence (QALCEGGHVFYNP) represented by positions 484 to 496 of SEQ ID NO: 2 or a modified sequence thereof, wherein the epitope either has a sequence comprising at least five consecutive amino acids including at least E, or a sequence that preserves the 487^{th} C and 489^{th} G when E is changed to Q.

### TECHNICAL FIELD

The present invention relates to immunological science specific for HHV-6B, and more particularly, relates to a neutralizing epitope thereof, screening of a therapeutic, and a medicament such as a vaccine thereof.

### BACKGROUND ART

HHV-6 is a strain separated from peripheral blood of an AIDS patient and a patient with a lymphoproliferative disorder, and is classified into β herpesviruses (T lymphotrophic herpesvirus) to which human herpesvirus 7 (HHV-7) and human cytomegalovirus (HCMV) belong.

HHV-6 can be classified into HHV-6A and HHV-6B as two kinds of variants. HHV-6B is thought to be a cause of exanthema subitum of infants. On the other hand, the relationship between HHV-6A and a disease in a human is unknown. The two kinds of variants are classified based on a difference in a nucleotide sequence, as well as by immunological and biological characteristics.

A main target of HHV-6 is thought to be a T cell line lymphocyte. HHV-6B latently-infects most of adults, and is a causative virus of exanthema subitum in the infant stage. Regarding HHV-6A, pathogenicity thereof has not been reported yet.

U97, U98, U99 and U100 which are genes of HHV-6A are reported to generate an mRNA transcript, which undergoes considerable splicing and encodes glycoproteins Q1 and Q2 (gQ1 and gQ2).

In cells infected with HHV-6, gQ1 binds to a gH/gL complex to form gH/gL/gQ1/gQ2. This tetrameric complex is found out in a virus envelope.

It is described that a gH/gL/gQ1/gQ2 complex of HHV-6A binds to human CD46, but the complex of HHV-6B does not bind thereto (Non-Patent Document 1: Journal of Virology, 2004, Vol. 78 (15) pp. 7969-7983; Non-Patent Document 2: Journal of Virology, 2003, Vol. 77 (4) pp. 2452-2458) (See FIG. 8). Non-Patent Document 1 (Journal of Virology, 2004, Vol. 78 (15) pp. 7969-7983) discloses that analysis regarding, intracellular processing with respect to gQ1 and gQ2 was performed. Non-Patent Document 2 (Journal of Virology, 2003, Vol. 77 (4) pp. 2452-2458) discloses that analysis regarding a 0100 gene product and analysis regarding formation of a complex with gH and gL were performed.

It is known that a neutralizing antibody under the name of gp105-82 (since gp105-82 is currently called gQ1, it is referred to as gQ1 in the present description) was made in HHV-6A. In addition, HHV-6A uses human CD46 as a cell receptor. It is still unknown whether a similar mechanism is adopted in HHV-6B or not, and a vaccine for the disease HHV-63 which is a cause of exanthema subitum and the like of infants and an effective method of screening other therapeutics have not been provided.
Non-Patent Document 3 (Cellular Microbiology (2009), 11 (7), 1001-1006) describes, for example, the relationship between human herpesvirus 6 (HHV-6) and CD46.
Non-Patent Document 4 (Journal of Virology, 1993, Vol. 67 (8) pp. 9611-4620) discloses mapping of gQ1.
Non-Patent Document 5 (Journal of Virology, 2009, Vol. 78 (9) pp. 4609-4616) discloses that formation of a complex of gH-gL and analysis regarding CD46 were performed.

### PRIOR ART DOCUMENTS

### Non-Patent Documents

Non-Patent Document 1: Journal of Virology, 2004, Vol. 78 (15) pp. 7969-7983
Non-Patent Document 2: Journal of Virology, 2003, Vol. 77 (4) pp. 2452-2458
Non-Patent Document 3: Cellular Microbiology (2009), 11 (7), 1001-1006
Non-Patent Document 4: Journal of Virology, 1993, Vol. 67 (8) pp. 4611-4620
Non-Patent Document 5: Journal of Virology, 2004, Vol. 78 (9) pp. 4609-4616

### SUMMARY OF THE INVENTION

### Problems to be Solved by the Invention

An object of the present invention is to provide a vaccine for the disease, wherein HHV-6B is the cause of exanthema subitum and the like in infants, which has not previously been provided, and to provide an effective method of screening other therapeutics.

### Solutions to the Problems

The present inventors have made intensive efforts and, as a result, solved the aforementioned problems by making a neutralizing monoclonal antibody (MAb) to HHV-6B, which is called KH-1. In the present invention, a HHB-6B protein recognized by this neutralizing antibody was also identified, and the antibody itself was characterized.

Accordingly, the present invention provides the followings:

(1) An epitope specific for HHV-6B, comprising a sequence of at least 5 consecutive amino acids comprising at least E, or comprising a sequence in which when E is changed to Q, C at the position 487 and G at the position 489 are conserved, among an amino acid sequence shown in the position 484 to the position 496 of SEQ ID No.: 2 (QALCEGGHVFYNP) or an, altered sequence thereof.
(2) The epitope according to item 1, comprising an amino acid sequence shown in the position 484 to the position 496 of SEQ ID No.: 2 (QALCEGGHVFYNP).
(3) An antibody to the epitope as defined in item 1 or 2 or an antigen binding fragment.
(4) The antibody or the antigen binding fragment according to item 3, having neutralizing activity.
(5) The antibody or the antigen binding fragment according to item 3 or 4, which is a monoclonal antibody.
(6) The antibody according to any one of items 3-5, comprising a light chain comprising a sequence shown in SEQ ID No.: 10 and a heavy chain comprising a sequence shown in SEQ ID No.: 12.
(7) An antigen comprising the epitope as defined in item 1 or 2.
(8) An antigen comprising the epitope as defined in item 1 or 2, comprising at least the position 1 to the position 496 of amino acids, among SEQ ID No.: 2 (full length of BgQ1).
(9) An antigen comprising the epitope as defined in item 1 or 2 comprising a full length BgQ1.
(10) A composition comprising the antigen as defined in any one of items 7-9.
(11) A composition for producing a neutralizing antibody of a HHV-6B virus, comprising the antigen as defined in any one of items 7-9.
(12) The composition according to item 11, wherein the antigen is HHV-6B gQ1.
(13) The composition according to item 11 or 12, further comprising HHV-6B gQ2.
(14) The composition according to item 13, wherein the HHV-68 gQ1 and the HHV-6B gQ2 have formed a complex.
(15) The composition according to item 13 or 14, wherein the HHV-6B gQ1 and the HHV-6B gQ2 are co-expressed in a cell.
(16) The composition according to any one of items 10-15, which is a medicament.
(17) The composition according to any one of items 10-16, which is a vaccine.
(18) A method of screening an inhibitor of a HHV-6B virus, the method comprising:

A) a step of providing HHV-6B gQ1 and HHV-6B gQ2;

B) a step of contacting a test substance with the HHV-6B gQ1 and the HHV-6B gQ2 under the condition in which the HHV-68 gQ1 and the HHV-6B gQ2 are bound; and

C) a step of observing binding between the HHV-6B gQ1 and the HHV-6B gQ2, wherein when the binding is inhibited, it is determined that the test substance is an inhibitor of a HHV-6B virus.

(19) The method according to item 18, wherein the HHV-6B gQ1 and the HHV-6B gQ2 are co-expressed in a cell.
(20) The method according to item 18 or 19, wherein gL and gH are further provided in the step A).
(21) A kit for screening an inhibitor of a HHV-6B virus, the kit comprising;

A) HHV-6B gQ1;

B) HHV-6B gQ2; and

C) a means for providing the condition under which the HHV-6B gQ1 and the HHV-6B gQ2 are bound, wherein

in the case where the binding is inhibited when a test substance is contacted with the HHV-6B gQ1 and the HHV-6B gQ2 under the condition in which the HHV-6B gQ1 and the HHV-6B gQ2 are bound, it is determined that the test substance is an inhibitor of a HHV-6B virus.

(22) The kit according to item 21, wherein the HHV-6B gQ1 and the HHV-6B gQ2 are co-expressed in a cell.
(23) The kit according to item 21 or 22, further comprising gL and gH.
(24) A method of screening a neutralizing epitope of a HHV-6B virus, the method comprising:

A) a step of providing an antibody comprising an antigen determining region (CDR) in SEQ ID No.: 10 and SEQ ID No.: 12 or an antigen binding fragment thereof;

B) a step of contacting a plurality of peptides being a candidate for the antibody or an antigen binding fragment thereof under the condition in which an epitope is bound; and

C) a step of determining a sequence having identity or similarity in the plurality of peptides bound to the antibody or an antigen binding fragment thereof, and selecting the sequence having identity or similarity as a neutralizing epitope.

(25) A kit for screening a neutralizing epitope of a HHV-6B virus, the kit comprising:

A) a means for providing an antibody comprising an antigen determining region (CDR) in SEQ ID No.: 10 and SEQ ID No.: 12 or an antigen binding fragment thereof;

B) a means for contacting a plurality of peptides being a candidate for the antibody or an antigen binding fragment thereof under the condition in which an epitope is bound; and

C) a means for determining a sequence having identity or similarity in the plurality of peptides bound to the antibody or an antigen binding fragment thereof, and selecting the sequence having identity or similarity as a neutralizing epitope.

The present inventors have immunized a mouse with a HHV-6B purified virion to make a monoclonal antibody to HHV-6B. A virus side factor recognized by an antibody obtained by immunoprecipitation was identified. Then, we have performed cloning of the identified virus side factor and preparation of a mutant thereof, and the mutant has been expressed in 293T cells, thereby, attempted to identify an epitope site of the present antibody, and we could obtain an antibody having the neutralizing activity on HHV-6B, and it has been revealed that the present antibody recognizes gQ1. When reactivity of the present antibody was investigated using a gQ1 mutant, it has been revealed that the present antibody recognizes amino acids at a C-terminal of gQ1. When C-terminal-deficient gQ1, and gQ2 which forms a complex with gQ1 have been co-expressed, and interaction therebetween has been investigated by immunoprecipitation and Western blotting, the mutant gQ1 deficient in amino acids recognized by the present antibody also has shown interaction with gQ2. From the foregoing, since a monoclonal antibody having the neutralizing activity which recognizes HHV-6B gQ1 was obtained, it was suggested that gQ1 plays an important role also upon entry of HHV-6B. Further, since a gQ1 mutant which is not recognized by the present antibody also showed interaction with gQ2, it was revealed that a gQ1 neutralizing epitope site and a region necessary for forming a complex with gQ2 are different. From these results, formation of a complex with gH and gL and further detailed analysis such as steric structure analysis of gQ1 using the present antibody can be performed.

The present inventors have succeeded in preparing a monoclonal antibody having the neutralizing activity on HHV-6B, gQ1 (glycoprotein Q1), and the present inventors have identified an epitope of the neutralization. It was discovered that this antibody reacts only with HHV-6B gQ1, and does not react with HHV-6A gQ1. That is, it appears that this neutralizing activity is HHV-6B-specific. It is known that gQ1 forms a complex with a glycoprotein named gH, gL and gQ2 in HHV-6A and binds to a receptor, but the receptor has not been identified in HHV-6B, and whether a complex is necessary or not is also unknown. In the result of the present invention, when gQ1 and gQ2 are expressed in a cell simultaneously, both are bound to each other.

It is known that a neutralizing antibody was prepared under the name of gp105-82 (currently, called gQ1) in HHV-6A (Non-Patent Document 7). In Non-Patent Document 7, it is not reported that formation of a complex with gQ1 and gQ2 is important. In addition, an epitope to be recognized is not reported. HHV-6A uses human CD46 as a cell receptor.

The present inventors thought theta further receptor for a variant of HHV-68 or both (HHV-6A and HHV-6B) plays an important role in determining cytotrophy of this virus. A glycoprotein in a virus envelope plays an essential role in virus infection, endplays an important role, particularly, in a process of virion entry. Further, since a glycoprotein provokes a neutralizing antibody, it serves as a main target of a host immune response. In the present invention, the present inventors separated a monoclonal antibody-producing hybridoma clone named KH-1. It was revealed that this clone has the neutralizing activity and has the ability to specifically react with HHV-6B gQ1.

HHV-6 enters a cell probably by an intracellular route. An envelope glycoprotein gH/gL/gQ1/gQ2 (gH/gL/gO) and gB functions in a process of virus adhesion and penetration. HHV-6A utilizes human CD46 as a cell receptor, but it was revealed in the present invention that HHV-6B would utilize another receptor unlike HHV-6A (FIG. 8).

### Advantages of the Invention

The present invention provides a medicament such as a vaccine for HHV-6B which is a cause of exanthema subitum, and a method of screening the medicament. In the present invention, it has been found out that the made monoclonal antibody recognizes HHV-6B gQ1, and the recognition becomes stronger when HHV-6A gQ1 is co-expressed with HHV-6B gQ2. That is, it is thought that a steric structure of formed gQ1 is recognized by the neutralizing antibody made in the present invention by the interaction between gQ1 and gQ2. A steric structure formed by binding between gQ1 and gQ2 serves as a target of HHV-6B infection neutralization. In addition, identification of a molecule which inhibits this binding can lead to development of a therapeutic.

### BRIEF DESCRIPTION OF THE DRAWINGS

FIG. 1 shows determination of a virus protein recognized by a monoclonal antibody to HHV-6B. Left two lanes show a monoclonal antibody BgQ202 used in immunoprecipitation, and right two lanes show KH-1. The leftmost lane and the lane second from the right show the result of a strain infected with HHV-6B, and the lane second from the left and the rightmost lane show a mock-infected strain.

FIG. 2 shows detection of gH/gL/gQ in a cell infected with HHV-6B by an anti-gQ1 monoclonal antibody. Left two lanes show immunoprecipitation with KH-1, and right three lanes show immunoprecipitation with a cell lysis product. The leftmost lane and the central lane show a mock-infected strain, the lanes second from the left and second from the right show a cell infected with HHV-6B, and the rightmost lane shows an experiment with a virion of HHV-6B. Numbers on the left side show the molecular weight (kDa).

FIG. 3 is the result showing that KH-1, being an anti-gQ1 antibody, has the neutralizing activity. The schematic view on the upper right side shows a scheme of an experiment of the present examples. The left side of the lower panel shows the result of a control of an indirect immunofluorescent assay, and the right side shows the result of the case where KH-1 was used.

FIG. 4 shows expression of a protein recognized by an antibody in a gQ1 transient expression system. The photograph on the left column shows BgQ1. The photograph on the right column shows BgQ1 and BgQ2. The panel on the upper row shows the result in the case where KH-1 was used as IFA. The panel on the lower row shows the result in the case where BgQ was used as IFA.

FIG. 5 shows a schematic diagram of various carboxy terminal-detection mutants of HHV-6B gQ1 gene and their reactivity with a monoclonal antibody KH-1. In FIG. 5, the reactivity of MAb antibody KH-1 with the various fragments of BgQ1 is indicated by + (reactive), and − (not reactive). Several fragments starting from the position 1 of HHV-6B gQ1 and continuing up to an amino acid position at the C-terminal are shown in the drawing.

FIG. 6(A) shows amino acid sequence alignment of gQ1 of HHV-6A and HHV-6B. The upper row shows the amino acid sequence of position 484 to position 496 of gQ1 of HHV-6B. The lower row shows the amino acid sequence of position 484 to position 496 of gQ1 of HHV-6A. FIG. 6(B) shows the result of confirmation of the presence or absence of the KH-1 reactivity of a point mutant at the C-terminal, for identification of a HHV-6B gQ1 epitope site recognized by KH-1. From the upper side, HHV-6B gQ1 (wild-type), E488Q (in which E at the position 488 was mutated to Q), C487Q E488Q (in which C at the position 487 was mutated to Q, and E at the position 488 was mutated to Q), and E488Q G489V (in which E at the position 488 was mutated to Q, and G at the position 488 was mutated to V).

FIG. 7 shows a model of inhibition of entry of HHV-6B by KH-1.

FIG. 8 is a schematic view showing a difference in the reactivity between HHV-6A and HHV-6B of a gH/gL/gQ1/gQ2 complex.

FIG. 9 is a schematic view of cell entry of HHV-6B. It is shown that gQ1 and gQ2 forma specific complex, information of a gH/gL/gQ1/gQ2 complex.

FIG. 10 is a schematic view showing a reaction of a neutralizing antibody and change in a steric structure. In the lower panel, amino acid sequences of a neutralizing epitope (HHV-6B) and HHV-6A corresponding thereto are shown.

### MODE FOR CARRYING OUT THE INVENTION

A preferable embodiment of the present invention will be explained below. It should be understood that, over the entirety of the present description, expression of a singular form, unless otherwise specified, also includes the concept of a plural form thereof. Therefore, it should be understood that an article of a singular form (e.g. “a”, “an”, and “the” in the case of English, and corresponding articles, adjectives and the like in other languages), unless otherwise specified, also includes the concept of a plural form thereof. In addition, it should be understood that terms used in the present description, unless otherwise specified, are used in a sense normally used in the art. Therefore, unless otherwise specified, all the technical terms and scientific and technological terms used in the present description have the same meaning as that generally understood by a person skilled in the art to which the present invention belongs. In the case of discrepancy, the present description (including the definition) prevails.

### DEFINITION

Definitions of terms which are particularly used in the present description will be listed below.

“HHV” used in the present description refers to a human herpesvirus, and there are type 1, type 2, type 3, type 4, type 5, type 6, type 7, type 8 and the like according to the type thereof.

As used herein, the term “herpesvirus” encompasses HHV-6A and HHV-6B including any type, and unless otherwise specified, encompasses both of a wild type and a recombinant type of these viruses. In addition, as used in the present description, the term “HHV-6 (human herpesvirus 6)” encompasses HHV-6A and HHV-6B, and unless otherwise specified, encompasses both of a wild type and a recombinant type of these viruses. HHV-6 belongs to the β subfamily like cytomegalovirus HHV-5, and HHV-6B is a causative virus of exanthema subitum, and it is stated that almost all people are infected therewith by 2 years old in Japan. Concerning HHV-6A, the relationship with a disease is not known.

As used herein, the “wild strain” of a herpesvirus refers to a herpesvirus strain isolated from nature, which has not undergone artificial alteration. Examples of the wild strain include a JI strain, but are not limited thereto.

As used herein, the “wild strain” of a herpesvirus such as HHV-6A or HHV-6B refers to a herpesvirus strain isolated from nature, which has not undergone artificial alteration (HHV-6A, HHV-6B etc.). Examples of the HHV-6A wild strain include a U1102 strain, but are not limited thereto. Examples of the HHV-6B wild strain include a HST strain, but are not limited thereto.

As used herein, a “mutant strain” of a herpesvirus such as HHV-6A or HHV-6B refers to a herpesvirus strain obtained by mutagenizing a virus strain being a wild strain by mutagenesis, many times of subculturing, or the like. When a herpesvirus strain is mutagenized, this mutagenesis may be random mutation introduction or site-specific mutation introduction.

As used herein, the “epitope” is used in a normal sense used in the art, and refers to a region determining antigenicity which is recognized by an antibody. An antibody, when it binds with a pathogenic microorganism or a polymeric substance, does not recognize the whole thereof but recognizes an epitope which is only a relatively small part of an antigen and binds thereto. The epitope is usually expressed by an amino acid sequence. In the case of a linear epitope, it is determined by an amino acid sequence of at least 5 amino acids, preferably at least 6 amino acids, 7 amino acids, or 8 amino acids. An antibody generated by entry of a particular antigen reacts only with one having an epitope which is identical with, or similar to that of the antigen.

As used herein, the “neutralizing epitope” refers to an epitope carrying out impartation of the neutralizing activity. By using an antigen having the neutralizing epitope, a vaccine can be produced, and therefore, an attention is paid to the antigen. The neutralizing epitope can be screened using, for example, a neutralizing antibody (e.g. KH-1 of the present invention (see SEQ ID Nos.: 10 and 12)).

As used herein, the “neutralizing activity” refers to the activity of inhibiting a subject such as a virus (representatively, a pathogen) from entering a cell or proliferating. The neutralizing activity is exerted and, as a result, pathogenicity is eliminated.

As used herein, concerning an immune reaction, “specific” refers to higher reactivity (preferably, the epitope reacts only with a subject) than the case of other subjects (e.g. antibody or antigen), and “specific for HHV-6B” refers to reactivity which is higher for HHV-6B than for HHV-6A (preferably, the epitope reacts only with HHV-6B). In addition, in the present description, an “epitope specific for HHV-6B” refers to an epitope having higher reactivity for HHV-6B than for HHV-6A (preferably, the epitope reacts only with HHV-6B).

As used herein, the “antibody” collectively refers to a protein which is produced in a living body by stimulation of an antigen and specifically binds to or reacts with an antigen in an immune reaction, or a protein having the same sequence as that of the protein which is produced by chemical synthesis or the like. An entity of the antibody is an immunoglobulin, and is also called Ab.

As used herein, an “antigen binding fragment” of an antibody refers to, concerning a certain antibody, a fragment having binding property on the same antigen as the antigen of the antibody. Whether a fragment is within the range of the “antigen binding fragment” or not can be assessed by an assay of affinity described in the present description. In the present description, such affinity can be shown using, as an index, a concentration at which an amount of binding of a labeled antigen to an antibody is inhibited by 50% (IC₅₀ value), and the IC₅₀ value can be calculated, for example, by a regression model with a logistic curve (Rodbard et al., Symposium on RIA and related procedures in medicine, P 165, Int. Atomic Energy Agency, 1974).

As used herein, an “anti-HHV-6B antibody” refers to an antibody which is provoked against HHV-6B or has the binding ability equivalent thereto. When the anti-HHV-6B antibody is mentioned, it is understood that, in order to retain the ability to bind to, an epitope (e.g. the neutralizing epitope of the present invention), an antibody in which a “heavy chain variable domain (VH)” and a “light chain variable (VH) domain” retain the particular binding ability is encompassed.

As used herein, the “neutralizing antibody” refers to any antibody having the neutralizing activity.

As used herein, the “heavy chain variable domain (VH)” and “light chain variable (VL) domain” of immunoglobulin are used in a sense normally used in the art. In an immunoglobulin, two L chains (light chain) and two H chains (heavy chain) having the same fundamental structure are connected with a S—S bond. In the H chain, two fragments of a Fc (crystallizable fragment) on a C-terminal side and a Fab (antigen binding fragment) on an N-terminal side are bent and connected at a hinge part, and a Y letter shape is taken as a whole. In both of the L chain and the H chain, in a sequence of about 110 amino acids (a length of about half of the L chain) from an N-terminal, chains are partially arranged in a different manner in accordance with antigen specificity. This part is called a variable part (a variable region, a V part), and variable parts (VL, VH) of both of the L chain and the H chain are related to determination of antigen specificity. A part other than a variable part is approximately constant for each class or every subclass, and is called a constant part (a constant region, a C part). In the constant part, one polypeptide unit consisting of about 110 amino acids (a homologous unit) (CL) in the L chain, three units (CH1, CH2, CH3) in IgG, IgA and IgD, and four units in IgM and IgE in the H chain are connected, and each unit or a region generated by binding with an opposite site is called a domain. The antibody of the present invention can be expressed using a part such as a domain.

As used herein, unless a different sense is particularly indicated, any polypeptide chain such as an antibody is described as having an amino acid sequence which starts at N-terminal extremity and ends at a C-terminal extremity. When an antigen binding site contains both V_{H} and V_{L} domains, these domains can be positioned at the same polypeptide molecule, and preferably, each domain can be positioned at a separate chain, and in this case, a V_{H} domain is a part of a heavy chain of an immunoglobulin, that is, an antibody or a fragment thereof, and V_{L} is a part of a light chain of an immunoglobulin, that is, an antibody or a fragment thereof. The antibody of the present invention can be expressed using a part such as the fragment.

Examples of an “antibody or antigen binding fragment” as used herein include an antibody and a chimeric antibody produced by a B cell or a hybridoma, a CDR-grafted antibody or a human antibody or an arbitrary fragment thereof, for example, F (ab′)₂ and Fab fragments, a single chain antibody and a single domain antibody. Therefore, the HHV-6B antibody or an antigen binding fragment can also be called a HHV-6B binding molecule, and it is understood that these include, for example, an antibody and a chimeric antibody produced by a B cell or hybridoma, a CDR-grafted antibody or a human antibody or an arbitrary fragment thereof, for example, F(ab′)₂ and Fab fragments, a single chain antibody and a single domain antibody, to which another molecule is bound.

The single chain antibody consists of variable domains of a heavy chain and a light chain of an antibody, which are covalently bound with a peptide linker consisting of 10 to 30 amino acids, preferably 15 to 25 amino acids. For this reason, the structure thereof does not include constant parts of a heavy chain and a light chain, and it is thought that a small peptide spacer has lower antigenicity than the whole constant part has. The “chimeric antibody” means an antibody in which a constant region of a heavy chain or a light chain or both of them is derived from a particular animal such as a human, while variable domains of both of a heavy chain and a light chain are derived from an animal other than the particular animal such as a human (e.g. a non-human-derived animal (e.g. a mouse) or another human), or are derived from a human but are derived from another human antibody. The “CDR-grafted antibody” means an antibody in which a hypervariable part region (CDR) is derived from a donor antibody such as a non-human (e.g. a mouse) antibody or another human antibody, while all or substantially all of other parts of an immunoglobulin, for example, a high preservation part of a constant region and a variable domain, that is, a framework region is derived from an acceptor antibody, for example, an antibody derived from a human. However, the CDR-grafted antibody contains a few amino acids of a donor sequence in a framework region, for example, a part of a framework region adjacent to a hypervariable region. The “humanized antibody” means an antibody in which all of constant and variable regions of both of a heavy chain and a light chain are derived from a human or are substantially the same as a human-derived sequence, and are not necessarily required to be derived from the same antibody, and which contains a mouse-produced antibody in which genes of mouse immunoglobulin variable part and constant part are replaced with a human counterpart, for example, one described in a general term in European patent No. 0546073B1, U.S. Pat. No. 5,545,806 or the like.

As used herein, “titer” refers to an amount of an antibody binding to an antigen that is contained in a unit volume of an anti-serum in a serum reaction. Actual measurement is performed by adding a constant amount of an antigen to a dilution series of an anti-serum, and a measured value is expressed in term of dilution-fold number at an end point at which a reaction occurs.

As used herein, affinity refers to a binding force between an antibody and a substance recognized by the antibody. In the present description, the affinity (K_{D}) is shown using, as an index, a dissociation constant of an antibody and a substance recognized by the antibody, such as an antigen. A method of measuring the affinity (K_{D}) is a common technical knowledge to a person skilled in the art, and for example, affinity can also be obtained by using a sensor chip.

The framework can be associated with any kind of a framework region, and preferably derived from a human. A suitable framework region can be selected by referring to the reference of Kabat E. A. et al. A preferable heavy chain framework is a human heavy chain framework. It can be determined from the information of a sequence of an antibody being a subject by referring to the aforementioned reference, and consists of sequences of FR1, FR2, FR3 and FR4 regions. By a similar method, an anti-HHV-6B light chain framework can be determined from the information of a sequence of an antibody being a subject by referring to the aforementioned reference, and consists of a sequence of FR1′, FR2′, FR3′ and FR4′ regions. The antibody of the present invention can be expressed by using a part such as the framework.

The terms “protein”, “polypeptide”, “oligopeptide” and “peptide” as used herein are herein used in the same meaning, and refer to a polymer of amino acids having any length. An antibody is usually one kind of a protein.

The term “polynucleotide”, “oligonucleotide” and “nucleic acid” as used herein are herein used in the same meaning, and refer to a polymer of nucleotides having an arbitrary length. Unless otherwise specified, it is intended that a particular nucleic acid sequence includes its conservatively modified altered body (e.g. a degenerate codon substitution body) and a complementary sequence, similarly to an explicitly shown sequence. Specifically, the degenerate codon substitution body can be accomplished by making a sequence in which a third position of selected one or more (or all) codons is substituted with a mixed base and/or a deoxyinosine residue (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)).

As used herein, a “gene” refers to a factor defining a genetic character. Genes are usually arranged on a chromosome in a certain order. A gene defining a primary structure of a protein is referred to as a structural gene, and a region influencing on its expression is referred to as a regulatory element. As used herein, the “gene” may refer to a “polynucleotide”, an “oligonucleotide” and a “nucleic acid” as well as/or a “protein”, a “polypeptide”, an “oligopeptide” and a “peptide”. As used herein, an “open reading frame” or “ORF” of a gene refers to a reading frame, which is one of three kinds of frames when a base sequence of a gene is cut by each three nucleotides and has an initiation codon, in which a termination codon does not appear midway and which has a some extent of a length, and has a possibility that it actually encodes a protein. In a herpesvirus genome, the whole base sequence thereof has been determined, at least 101 genes have been identified, and it is known that each of the genes has an open reading frame (ORF).

As used herein, gQ refers to a glycoprotein. In HHV-6B, a gQ gene encodes a 37 kDa glycoprotein and is derived from an alternative splicing transcript.

When “HHV-6B gQ1” or HHV-6B used herein is referred to, mere “gQ1” (gene) is a molecule also called gp105-82 and is also found out in NC_{—}000898 (genome sequence) in GenBank. Specifically, gQ1 has a sequence shown in SEQ ID No.: 2 or an altered body thereof, and for example, a protein thereof can be:

(a) a polypeptide consisting of an amino acid sequence shown in SEQ ID No.: 2 or a fragment thereof;

(b) a polypeptide having an amino acid sequence shown in SEQ ID No.: 2 in which one or more amino acids have at least one mutation selected from the group consisting of substitution, addition and deletion, and having the biological activity;

(c) a polypeptide encoded by a splicing mutant or an allele mutant of a base sequence encoding SEQ ID No.: 2;

(d) a polypeptide which is a species homolog of an amino acid sequence shown in SEQ ID No.: 2;

(e) a polypeptide having an amino acid sequence having identity with any one polypeptide of (a) to (d) of at least 70%, and having the biological activity; or

(f) a polypeptide having an amino acid sequence encoded by a polynucleotide which hybridizes with a polynucleotide encoding any one polypeptide of (a) to (d) under the stringent hybridization condition, and having the biological activity.

When “HHV-6B gQ2” or HHV-6B used herein is referred to, mere “gQ2” (gene) interacts with a gH/gL/gQ1 complex in a cell infected with HHV-6 or a virion, and is also found out in NC 000898 (genome sequence) in GenBank. Specifically, “gQ2” has a sequence shown in SEQ ID No.: 4 or an altered body thereof, and for example, a protein thereof can be:

(a) a polypeptide consisting of an amino acid sequence shown in SEQ ID No.: 4 or a fragment thereof;

(b) a polypeptide consisting of an amino acid sequence shown in SEQ ID No.: 4 in which one or more amino acids have at least one mutation selected from the group consisting of substitution, addition and deletion, and having the biological activity;

(c) a polypeptide encoded by a splicing mutant or an allele mutant of a base sequence encoding SEQ ID No.: 4;

(d) a polypeptide which is a species homolog of an amino acid sequence shown in SEQ ID No 4;

(e) a polypeptide having an amino, acid sequence having identity with any one polypeptide of (a) to (d) of at least 70%, and having the biological activity; or

(f) a polypeptide having an amino acid sequence encoded by a polynucleotide which hybridizes with a polynucleotide encoding any one polypeptide of (a) to (d) under the stringent hybridization condition, and having the biological activity.

When “HHV-6B gH” or HHV-6B used herein is referred to, mere “gH” (gene) is one molecule forming a gH/gL/gQ1/gQ2 complex in a cell infected with HHV-6 or a virion, and is also found out in NC_{—}000898 (genome sequence) in GenBank. Specifically, “gH” has a sequence shown in SEQ ID No.: 6 or an altered body thereof, and for example, a protein thereof can be:

(a) a polypeptide consisting of an amino acid sequence shown in SEQ ID No.: 6 or a fragment thereof;

(b) a polypeptide consisting of an amino acid sequence shown in SEQ ID No.: 6 in which one or more amino acids have at least one mutation selected from the group consisting of substitution, addition and deletion, and having the biological activity;

(c) a polypeptide encoded by a splicing mutant or an allele mutant of a base sequence encoding SEQ ID No.: 6;

(d) a polypeptide which is a species homolog of an amino acid sequence shown in SEQ ID No.: 6;

(e) a polypeptide having an amino acid sequence having identity with any one polypeptide of (a) to (d) of at least 70%, and having the biological activity; or

(f) a polypeptide having an amino acid sequence encoded by a polynucleotide which hybridizes with a polynucleotide encoding any one polypeptide of (a) to (d) under the stringent hybridization condition, and having the biological activity.

When “HHV-6B gL” or HHV-6B used herein is referred to, mere “gL” (gene) is one molecule forming a gH/gL/gQ1/gQ2 complex in a cell infected with HHV-6 and in a virion, and is also found out in NC_{—}000898 (genome sequence) in GenBank. Specifically, “gL” has a sequence shown in SEQ ID No.: 8 or an altered body thereof, and for example, a protein thereof can be:

(a) a polypeptide consisting of an amino acid sequence shown in SEQ ID No.: 8 or a fragment thereof;

(b) a polypeptide consisting of an amino acid sequence shown in SEQ ID No.: 8 in which one or more amino acids have at least one mutation selected from the group consisting of substitution, addition and deletion, and having the biological activity;

(c) a polypeptide encoded by a splicing mutant or an allele mutant of a base sequence encoding SEQ ID No.: 8;

(d) a polypeptide which is a species homolog of an amino acid sequence shown in SEQ ID No.: 8;

(e) a polypeptide having an amino acid sequence having identity with any one polypeptide of (a) to (d) of at least 70%, and having the biological activity; or

(f) a polypeptide having an amino acid sequence encoded by a polynucleotide which hybridizes with a polynucleotide encoding anyone polypeptide of (a) to (d) under the stringent hybridization condition, and having the biological activity.

As used herein, the “corresponding” amino acid and nucleic acid refer to an amino acid and a nucleic acid which have the same actions as the predetermined amino acid and nucleic acid in a polypeptide and a nucleic acid molecule being a standard of comparison, or are expected to have the above actions, in a certain polypeptide and a certain nucleic acid molecule, respectively. For example, concerning gQ1, gQ2, gL, gH or the like, the “corresponding” amino acid and nucleic acid refer to sequences which are aligned and correspond when alignment is performed in corresponding genes (amino acid, nucleic acid etc.), in other mutants or the like, regarding HHV-6B or the like. For example, the “corresponding” amino acid and nucleic acid are an amino acid which is present at the same position as a position of a certain standard and which contributes to the catalytic activity, and a nucleic acid encoding the same, respectively. For example, in the case of a nucleic acid sequence, the “corresponding” nucleic acid can be that nucleic acid sequence or a part exerting the same function as that of a particular part encoded by the sequence.

As used herein, the “corresponding” gene (e.g. a polypeptide or a nucleic acid molecule) refers to a gene which has the same action as that of a predetermined gene in a species being a standard of comparison, or is expected to have that action. In the case where a plurality of genes having such action are present, the “corresponding” gene refers to genes having the same evolutionary origin. Therefore, a corresponding gene of a certain gene can be an ortholog of the gene. Therefore, a sequence of a herpesvirus type 6B and a gene corresponding to a gene of a cancer antigen or the like can also be found out in other organisms (other mutant strains of herpesvirus 6B, herpesvirus type 7 etc.). Such a corresponding gene can be identified using a technique well-known in the art. Therefore, for example, a corresponding gene in a certain animal can be found out by searching sequence database of an organism or a virus (e.g. herpesvirus 6B) using, as a query sequence, a sequence of a gene being a standard of a corresponding gene (e.g. gQ1, gQ2, gL, gH sequence etc. of herpesvirus-6A), or screening a library by a wet experiment.

As used herein, the “isolated” substance (e.g. a biological factor such as a nucleic acid or a protein) refers to a substance substantially, separated or purified from other substances (preferably, a biological factor) (for example, in the case of a nucleic acid, a factor other than a nucleic acid and a nucleic acid containing a nucleic acid sequence other than an objective nucleic acid; in the case of a protein, a factor other than a protein and a protein containing an amino acid sequence other than an objective protein etc.) of the environment in which the substance is naturally present (e.g. in, a cell of an organism body). The “isolated” nucleic acid and protein include a nucleic acid and a protein which are purified by a standard purification method. Therefore, the isolated nucleic acid and protein, include chemically synthesized nucleic acids and proteins.

As used herein, the “purified” substance (e.g. a biological factor such as a nucleic acid or a protein) refers to a substance from which at least a part of a factor naturally accompanying with the substance has been removed. Therefore, usually, the purity of a substance in the purified substance is higher than that in the usual state in which the substance exists (that is, the substance is concentrated).

As used herein, “purified” and “isolated” mean that preferably at least 75% by weight, more preferably at least 85% by weight, further preferably at least 95% by weight, and most preferably at least 98% by weight of the same type of a substance exists.

As used herein, “homology” of a gene refers to a degree of identity to each other of two or more sequences (e.g. amino acid sequence and nucleic acid sequence). Therefore, as homology between certain two sequences (e.g. between wild type and altered body) is higher, identity or similarity of the sequences is higher. Whether two kinds of sequences have homology or not can be investigated by direct comparison of sequences, or in the case of a nucleic acid, by a method of hybridization under the stringent condition. In the case where two sequences are directly compared, when sequences are identical between the sequences representatively by at least 50%, preferably by at least 70% identical, more preferably by at least 80%, 90%, 95%, 96%, 97%, 98% or 99% identical, these sequences have homology.

As used herein, the “stringent hybridization condition” refers to the well-known condition which is conventionally used in the art. Such a polynucleotide can be obtained by a colony hybridization method, a plaque hybridization method or a Southern blot hybridization method employing a polynucleotide selected from the polynucleotides of the present invention as a probe. Specifically, a polynucleotide which hybridizes under the stringent condition means a polynucleotide which can be identified by performing hybridization at 65° C. in the presence of 0.7 to 1.0 M NaCl using a filter with a colony or plaque-derived DNA immobilized thereon, and washing a filter under the condition of 65° C. using a SSC (saline-sodium citrate) solution having a 0.1 to 2-fold concentration (the composition of a SSC solution of a 1-fold concentration is 150 mM sodium chloride, 15 mM sodium citrate). Hybridization can be performed in accordance with the method described in an experimental document such as Molecular Cloning 2^{nd} ed., Current Protocols in Molecular Biology, Supplement 1-38, DNA Cloning 1: Core Techniques, A Practical Approach, Second Edition, Oxford University Press (1995). Herein, from sequences which hybridize under the stringent condition, preferably, sequences containing only an A sequence or only a T sequence are excluded. A “hybridizable polynucleotide” refers to a polynucleotide which can hybridize with another polynucleotide under the aforementioned hybridizing condition. Specific examples of the hybridizable polynucleotide include a polynucleotide having 60% or more homology, preferably a polynucleotide having 80% or more homology, further preferably a polynucleotide having 95% or more homology with a base sequence of a DNA encoding a polypeptide having an amino acid sequence specifically shown in the present invention.

Comparison of identity of, and calculation of homology of base sequences are herein performed using default parameters employing BLAST which is a tool for sequence analysis. Search of identity can be performed using BLAST 2.2.9 of NCBI (published on May 12, 2004), for example. Values of identity in the present description usually refer to values obtained by performing alignment under the default condition using the BLAST, provided that the highest value is adopted as a value of identity when a higher value is obtained by change in parameters. When identity is assessed in a plurality of regions, the highest value among them is adopted as a value of identity.

As used herein, “search” refers to finding of other nucleic acid base sequences having particular function and/or nature utilizing a certain nucleic acid base sequence by, for example, an electronic or biological method. Examples of the electronic search include BLAST (Altschul et al., J. Mol. Biol. 215: 403-410 (1990)), FASTA (Pearson & Lipman, Proc. Natl. Acad. Sci., USA 85: 2444-2448 (1988)), Smith and Waterman method (Smith and Waterman, J. Mol. Biol. 147: 195-197 (1981)), and Needleman and Wunsch method (Needleman and Wunsch, J. Mol. Biol. 48: 443-453 (1970)), but are not limited thereto. Examples of the biological search include stringent hybridization, a microarray in which a genome DNA is adhered to a nylon membrane or the like or a microarray in which a genome DNA is adhered to a glass plate (microarray assay), PCR and in situ hybridization, but are not limited thereto. In the present description, it is intended that gQ1, gQ2, gL, gH and the like used in the present invention should include such corresponding sequences identified by electronic search or biological search.

An amino acid can be herein referred to by three letters symbols which are generally known, or one letter symbols which are recommended by IUPAC-IUB Biochemical Nomenclature Commission. A nucleotide can be similarly referred to by a one letter code which is generally accepted.

As used herein, the “fragment” refers to a polypeptide or a polynucleotide having a sequence length of 1 to n−1, relative to a full length polypeptide or polynucleotide (length is n). The length of the fragment can be appropriately changed according to the purpose thereof. Examples of a lower limit of the length thereof, in the case of a polypeptide, include 3, 4, 5, 6, 7, 8, 9, 10, 15, 20, 25, 30, 40, 50 and more amino acids, and a length represented by an integer which is not specifically listed herein (e.g. 11) can also be proper as a lower limit. In addition, in the case of a polynucleotide, examples of a lower limit of the length thereof include 5, 6, 7, 8, 9, 10, 15, 20, 25, 30, 40, 50, 75, 100, 200, 300, 400, 500, 600, 700, 800, 900, 1000 and more nucleotides, and a length represented by an integer which is not specifically listed herein (e.g. 11) can also be proper as a lower limit.

A polypeptide used in the present invention may be one in which one or more (e.g. one or a few) amino acids in an amino acid sequence may be substituted, added and/or deleted, or a sugar chain may be substituted, added and/or deleted, as far as it has substantially the same action (e.g. neutralizing activity) as that of a natural polypeptide.

It is well-known in the art that a certain amino acid is substituted with another amino acid having a similar hydrophobicity index, thereby, a protein still having a similar biological function (e.g. a protein having an equivalent enzyme activity) can be generated. In such amino acid substitution, the hydrophobicity index is preferably within ±2, more preferably within ±1, and further preferably within ±0.5. It is understood in the art that substitution of an amino acid based on hydrophobicity is efficient. A hydrophilicity index is also considered in preparing an altered body. As described in U.S. Pat. No. 4,559,101, the following hydrophilicity indices are assigned to amino acid residues: arginine (+3.0); lysine (+3.0); aspartic acid (+3.0±1); glutamic acid (+3.0±1);

serine (+0.3); asparagine (+0.2); glutamine (+0.2); glycine (0); threonine (−0.4); praline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that a protein can be substituted with another protein in which an amino acid has a similar hydrophilicity index and which can still give a biologically equivalent body. In such amino acid substitution, the hydrophilicity index is preferably within ±2, more preferably within ±1, and further preferably within ±0.5.

In the present invention, “conservative substitution” refers to substitution in which the hydrophilicity index or/and the hydrophobicity index of the original amino acid and a substituting amino acid are similar as described above, in amino acid substitution. In the present description, “similar substitution” refers to that the hydrophilicity index is within ±2. Examples of the conservative substitution are well-known to a person skilled in the art, and include substitution within each of the following groups, but are not limited thereto: arginine and lysine; glutamic acid and aspartic acid; serine and threonine; glutamine and asparagine; as well as valine, leucine, and isoleucine.

As used herein, the “altered body” refers to an entity in which a part is changed relative to a substance such as the original polypeptide or polynucleotide. Examples of such an altered body include a substitution altered body, an addition altered body, a deletion altered body, a truncated altered body, and an allele mutant. The allele refers to genetic altered bodies which belong to the same locus and are discriminated from each other. Therefore, the “allele mutant” refers town altered body having the relationship of an allele relative to a certain gene. The “species homolog or a homolog” refers to an entity having homology (preferably 60% or more homology, more preferably 80% or more, 85% or more, 90% or more, 95% or more homology) with a certain gene, at the level of amino acid or nucleotide, in a certain species. A method of obtaining such a species homolog is apparent from the description of the present description. The “ortholog” is also referred to as an orthologous gene, and refers to two genes that are derived from species differentiation from a common ancestor. For example, using a hemoglobin gene family having a multigenic structure, human and mouse a hemoglobin genes are orthologs, but a human α hemoglobin gene and a human β hemoglobin gene are paralogs (genes generated from gene overlapping). Since the ortholog is useful for presuming a molecular genealogical tree, the ortholog of the present invention can also be useful in the present invention.

As used herein, the “functional altered body” refers to an altered body retaining the biological activity (particularly, neutralizing activity) born by a sequence being a standard.

As used herein, a “conservatively (altered) altered body” is applied to both of an amino acid sequence and a nucleic acid sequence. Regarding a particular nucleic acid sequence, the altered body which was conservatively altered refers to a nucleic acid encoding the same or essentially the same amino acid sequence, and when a nucleic acid does not encode an amino acid sequence, refers to essentially the same sequence. Due to degeneracy of a genetic code, many functionally same nucleic acids encode an arbitrary predetermined protein. For example, all of codons GCA, GCC, GCG, and GCU encode an amino acid alanine. Therefore, in all positions in which alanine is specified by a codon, the codon can be changed into any of described corresponding codons, without changing an encoded polypeptide. Such a variation in a nucleic acid is “silent alteration (mutation)” which is one kind of conservatively altered mutations. In a nucleic acid, conservative substitution can be confirmed, for example, while the neutralizing activity is measured.

As used herein, in order to, prepare a gene encoding a functionally equivalent polypeptide, in addition to substitution of an amino acid, addition, deletion or modification of an amino acid can also be conducted. Substitution of an amino acid refers to substitution of the original peptide with one or more, for example, 1 to 10, preferably 1 to 5, more preferably 1 to 3 amino acids. Addition of an amino acid refers to addition, of one or more, for example, 1 to 10, preferably 1 to 5, more preferably 1 to 3 amino acids to the original peptide chain. Deletion of an amino acid refers to deletion of one or more, for example, 1 to 10, preferably 1 to 5, more preferably 1 to 3 amino acids from the original peptide. The amino acid modification includes amidation, carboxylation, sulfation, halogenation, alkylation, glycosylation, phosphorylation, hydroxylation, and acylation (e.g. acetylation), but are not limited thereto. An amino acid to be substituted or added may be a natural amino acid, a non-natural amino acid, or an amino acid analog. A natural amino acid is preferable.

A nucleic acid encoding a polypeptide such as the antigen of the present invention can be obtained by a well-known PCR method, or can be chemically synthesized. These methods may be combined with, for example, a site-specific mutagenesis method or a hybridization method.

As used herein, “substitution, addition or deletion” of a polypeptide or a polynucleotide refers to substitution, addition or removal of an amino acid or a substitute thereof, or a nucleotide or a substitute thereof, relative to the original polypeptide or polynucleotide. The technique of such substitution, addition or deletion is well-known in the art, and examples of such a technique include a site-specific mutagenesis technique. The number of substitution, addition or deletion may be any number as far as it is one or more, and such a number can be made larger as far as an objective function is retained in an altered body having the substitution, addition or deletion. For example, such a number can be one or a few, and preferably within 20%, within 10% of a total length, or can be 100 or less, 50 or less, 25 or less or the like.

As used herein, “screening” refers to selection of a factor such as a substance having an objective particular nature from many candidates by a particular manipulation and/or assessing method. In the present invention, it is understood that a factor such as a substance having the desired activity obtained by screening is also included in the scope of the present invention.

As used herein, an “effective amount” of a vaccine, a drug or the like refers to an amount with which the drug or the like can exert the objective drug efficacy. In the present description, of such an effective amount, the minimum concentration is sometimes referred to as the minimum effective amount. Such a minimum effective amount is well-known in the art and, usually, a minimum effective amount of a drug or the like has been determined by a person skilled in the art or can be appropriately determined by a person skilled in the art. In determining such an effective amount, it is possible to use an animal model besides actual administration. The present invention is also useful upon determination of such an effective amount. In the present invention, an effective amount of a vaccine or the like can also be appropriately determined.

As used herein, the “pharmaceutically acceptable carrier” refers to a substance which is used when a medicament is produced and which does not give any adverse influence on an active ingredient. Examples of such a pharmaceutically acceptable carrier include antioxidants, preservatives, coloring materials, flavors, diluents, emulsifiers, suspending agents, solvents, fillers, bulking agents, buffers, delivery vehicles, excipients and/or agricultural or pharmaceutical adjuvants, but are not limited thereto.

The kind and the amount of the drug or the like used in the treatment method of the present invention can be easily determined by a person skilled in the art based on information obtained by the method of the present invention (e.g. information regarding a disease) in view of a use purpose, a subject disease (kind, severity etc.), age, weight, sex, and health history of a patient, form or kind of a site of a subject to receive administration of the drug, and the like. The frequency of application of the monitoring method of the present invention to a subject (or a patient) can also be easily determined by a person skilled in the art in view of a use purpose, a subject disease (kind, severity etc.), age, weight, sex, and health history of a patient, and a therapeutic process. Examples of the frequency of monitoring the disease state include monitoring on every day to once per a few months (e.g. once per one week to once per one month). It is preferable that monitoring of once per one week to one month is applied while following the course.

As used herein, the “instruction” is a description of a method of administering a medicament or the like of the present invention or a method of diagnosis, a method of treatment of the present invention or the like for a person performing administration such as a doctor, a patient or the like and a person performing diagnosis (which may be a patient himself/herself), an implementer such as a person performing screening and the like. This instruction includes descriptions instructing a method of using a diagnostic, a preventive, a medicament or the like of the present invention, for example, the number of times, interval or the like of administration of a vaccine. This instruction is produced according to the formality specified by the supervisory authority in a country where the present invention is worked (for example, Ministry of Health, Labour and Welfare in Japan, Food and Drug Administration (FDA) in USA etc.), and the fact that an approval was issued from the supervisory authority is explicitly described. The instruction is a so-called package insert and is usually provided on a paper medium, but is not limited thereto. For example, the instruction can be provided in a form such as a film adhered to a bottle and an electronic medium (e.g. a homepage provided on the internet (website) and electronic mail).

If necessary, in the treatment of the present invention, two or more kinds of drugs or the like can be used. When two or more kinds of drugs or the like are used, substances having similar natures or origins may be used, or drugs or the like having different natures or origins may be used. Information regarding a disease level for such a method of administering two or more kinds of drugs or the like can be obtained by the method of the present invention.

A culturing method used in the present invention is described and supported by, for example, Animal Cultured Cell Manual, edited and authored by Seno et al., Kyoritsu Shuppan Co., Ltd., 1993, and all descriptions thereof are incorporated in the present description.

### (Process for Producing Polypeptide)

The antigen, the vaccine or the like of the present invention can be a polypeptide. Such a polypeptide can be produced by culturing a transformant derived from a microorganism, an animal cell or the like harboring a recombinant vector in which a DNA encoding the polypeptide (antigen etc.) of the present invention is incorporated according to a normal culturing method to generate and accumulate the polypeptide of the present invention, and collecting the polypeptide of the present invention from the culture of the present invention.

A method of culturing the transformant of the present invention in a medium can be performed according to a normal method used in culturing a host. As a medium for culturing a transformant obtained by using a prokaryote such as Escherichia coli or a eukaryote such as yeast as a host, any of a natural medium and a synthetic medium may be used as far as it is a medium which contains a carbon source, a nitrogen source, inorganic salts and the like which can be utilized by the organism of the present invention and in which culturing of a transformant can be efficiently performed.

As the carbon source, a carbon source which can be utilized by each microorganism may be used, and carbohydrates such as glucose, fructose, sucrose, molasses containing them, starch, and a starch hydrolysate, organic acids such as acetic acid and propionic acid, and alcohols such as ethanol and propanol can be used.

As the nitrogen source, ammonia, ammonium salts of various inorganic acids or organic acids such as ammonium chloride, ammonium sulfate, ammonium acetate, and ammonium phosphate, other nitrogen-containing substances, as well as peptone, meat extract, yeast extract, corn steep liquor, casein hydrolysate, soybean cake and soybean cake hydrolysate, various fermentation microorganisms and digestion products thereof can be used.

As the inorganic salt, primary potassium phosphate, secondary potassium phosphate, magnesium phosphate, magnesium sulfate, sodium chloride, ferrous sulfate, manganese sulfate, copper sulfate, calcium carbonate and the like can be used. Culturing is performed under an aerobic condition such as in shaking culturing or deep aeration stirring culturing.

The culturing temperature is suitably 15 to 40° C., and the culturing time is usually 5 hours to 7 days. During culturing, the pH is retained at 3.0 to 9.0. Adjustment of the pH is performed using an inorganic or organic acid, an alkali solution, urea, calcium carbonate, ammonia or the like. Alternatively, during culturing, if necessary, an antibiotic such as ampicillin or tetracycline may be added to a medium.

When a microorganism transformed with an expression vector using an inducible promoter is cultured, if necessary, an inducer may be added to a medium. For example, when a microorganism transformed with an expression vector using a lac promoter is cultured, isopropyl-β-D-thiogalactopyranoside or the like may be added to a medium, and when a microorganism transformed with an expression vector using a trp promoter is cultured, indoleacrylic acid or the like may be added to a medium. A plant cell or organ with a gene introduced therein can be cultured in a large scale using a jar fermenter. As a medium in which culturing is performed, the Murashige and Skoog (MS) medium and the White medium, which are generally used, or a medium obtained by adding a plant hormone such as auxin, cytokine or the like to the above medium can be used.

For example, when an animal cell is used, as a medium in which the cell of the present invention is cultured, the RPMI 1640 medium [The Journal of the American Medical Association, 199, 519 (1967)], the Eagle's MEM medium [Science, 122, 501 (1952)], the DMEM medium [Virology, 8, 396 (1959)], and the 199 medium [Proceedings of the Society for the Biological Medicine, 73, 1 (1950)] which are generally used, or a medium obtained by adding bovine fetal serum or the like to the above medium are used.

Culturing is usually performed for 1 to 7 days under the conditions of a pH of 6 to 8, 25 to 40° C., under the presence of 5% CO₂. In addition, during culturing, if necessary, an antibiotic such as kanamycin, penicillin, or streptomycin may be added to a medium.

In order to isolate or purify the polypeptide of the present invention from a culture of a transformant transformed with a nucleic acid sequence encoding the polypeptide of the present invention, a normal method of isolating or purifying an enzyme which is well-known and conventionally used in the art can be used. For example, when the polypeptide of the present invention is secreted to the outside of cells of a transformant for producing the polypeptide of the present invention, the culture is treated by a procedure such as centrifugation to obtain a soluble fraction. From the soluble fraction, a purified authentic sample can be obtained using a procedure such as a solvent extraction method, a salting out method and a desalting method with ammonium sulfate or the like, a precipitation method with an organic solvent, an anion exchange chromatography method using a resin such as diethylaminoethyl (DEAE)-Sepharose, DIAION HPA-75 (Mitsubishi Chemical Corporation) or the like, a cation exchange chromatography method using a resin such as S-Sepharose FF (Pharmacia) or the like, a hydrophobic chromatography method using a resin such as butyl Sepharose, phenyl Sepharose or the like, a gel filtration method using a molecular sieve, an affinity chromatography method, a chromatofocusing method, or an electrophoresis method such as isoelectric focusing.

When the polypeptide of the present invention is accumulated in cells of a transformant for producing the polypeptide of the present invention in the dissolved state, cells in the culture are collected by centrifuging the culture, the cells are washed, and the cells are crushed by an ultrasound crushing machine, a French press, a Manton Gaulin homogenizer, a dino-mill or the like to obtain a cell-free extract. A purified authentic sample can be obtained from the supernatant obtained by centrifuging the cell-free extract by using a procedure such as a solvent extraction method, a salting out method and a desalting method with ammonium sulfate or the like, a precipitation method with an organic solvent, an anion exchange chromatography method using a resin such as diethylaminoethyl (DEAE)-Sepharose, DIATOM HPA-75 (Mitsubishi Chemical Corporation) or the like, a cation exchange chromatography method using a resin such as S-Sepharose FF (Pharmacia) or the like, a hydrophobic chromatography method using a resin such as butyl Sepharose or phenyl Sepharose, a gel filtration method using a molecular sieve, an affinity chromatography method, a chromatofocusing method, or an electrophoresis method such as isoelectric focusing.

In addition, when the polypeptide of the present invention is expressed by forming insolubles in cells, similarly, cells are recovered, crushed, and centrifuged, and from the resulting precipitated fraction, the polypeptide of the present invention is recovered by a normal method, and thereafter, the insolubles of the polypeptide are solubilized with a polypeptide denaturing agent. This solubilized liquid is diluted in a solution which does not contain a polypeptide denaturing agent or in which the concentration of a polypeptide denaturing agent is dilute to such an extent that the polypeptide is not denatured, or dialyzed to constitute the polypeptide of the present invention into a normal steric structure, and a purified authentic sample can be obtained by an isolating and purifying method which is the same as that described above.

Alternatively, the polypeptide can be purified in accordance with a normal method of purifying a protein [J. Evan. Sadler et al.: Methods in Enzymology, 83, 458]. Alternatively, the polypeptide of the present invention can be produced as a fused protein with another protein, and this can be purified by utilizing affinity chromatography using a substance having affinity for the fused protein [Akio Yamakawa, Experimental Medicine, 13, 469-474 (1995)]. For example, in accordance with the method described in the method of Lowe et al. [Proc. Natl. Acad. Sci., USA, 86, 8227-8231 (1989), Genes Develop., 4, 1288 (1990)], the polypeptide of the present invention can be produced as a fused protein with Protein A, and can be purified by affinity chromatography using immunoglobulin G.

Alternatively, the polypeptide of the present invention can be produced as a fused protein with the FLAG peptide, and can be purified by affinity chromatography using an anti-FLAG antibody [Proc. Natl. Acad. Sci., USA, 86, 8227 (1989), Genes Develop., 4, 1288 (1990)].

Further, the polypeptide of the present invention can also be purified by affinity chromatography using an antibody to the polypeptide of the present invention itself. The polypeptide of the present invention can be produced using an in vitro transcription and translation system in accordance with a known method [J. Biomolecular NMR, 6, 129-134, Science, 242, 1162-1164, J. Biochem., 110, 166-168 (1991)].

The polypeptide of the present invention can also be produced by a chemical synthesis method such as the Fmoc method (fluorenylmethyloxycarbonyl method) or the tBoc method (t-butyloxycarbonyl method) based on amino acid information of the polypeptide obtained above. Alternatively, the polypeptide of the present invention can also be chemically synthesized utilizing a peptide synthesizer of Advanced ChemTech, Applied Biosystems, Pharmacia Biotech, Protein Technology Instrument, Synthecell-Vega, PerSeptive, Shimadzu Corporation or the like.

Structural analysis of the purified polypeptide of the present invention can be carried out by a method which is usually used in protein chemistry, for example, the method described in Protein Structural Analysis for Gene Cloning (authored by Hisashi Hirano, published by Tokyo Kagaku Dojin, 1993).

Deletion, substitution or addition of an amino acid of the polypeptide of the present invention can be carried out by a site-specific mutagenesis method which is a technique well-known before filing. Such deletion, substitution or addition of one or a few amino acids can be prepared in accordance with the methods described in Molecular Cloning, A Laboratory Manual, Second Edition, Cold Spring Harbor Laboratory Press (1989), Current Protocols in Molecular Biology, Supplement 1-38, John Wiley & Sons (1987-1997), Nucleic Acids Research, 10, 6487 (1982), Proc. Natl. Acad. Sci., USA, 79, 6409 (1982), Gene, 34, 315 (1985), Nucleic Acids Research, 13, 4431 (1985), Proc. Natl. Acad. Sci., USA, 82, 488 (1985), Proc. Natl. Acad. Sci., USA, 81, 5662 (1984), Science, 224, 1431 (1984), PCT WO 85/00817 (1985), Nature, 316, 601 (1985) and the like.

### (Immunotherapy)

As used herein, a “vaccine” usually refers to a composition (e.g. a suspension or a solution) containing an infective factor or a part having an infection factor, or a factor which can produce such a factor or part (e.g. a gene sequence), which is administered into a body to generate active immunity. An antigenic part constituting the vaccine can be a microorganism (e.g. a virus or a bacterium) a natural product purified from a microorganism, a synthetic product, a protein, a peptide, a polysaccharide or a similar product obtained by genetic manipulation, or a nucleic acid molecule containing a nucleic acid sequence encoding such a protein. The vaccine manifests its effect by giving rise to a neutralizing antibody. The vaccine may be a gene vaccine, and the gene vaccine refers to, among the vaccines, a composition (e.g. a suspension or a solution) containing a factor which is expressed in a subject to which the factor is administered and in which the expression product has an action of the vaccine (representatively, a nucleic acid molecule). A representative gene vaccine can be a nucleic acid molecule (e.g. a vector, a plasmid, or a Naked DNA) containing a nucleic acid sequence encoding a gene product having antigenicity.

In the present description, the immunological effect of the vaccine can be confirmed using any method known in the art. Examples of such a method include CTL precursor cell frequency analysis, an ELISPOT method, a tetramer method, and a real time PCR method, but are not limited thereto. As illustrative explanation, in the CTL precursor cell frequency analysis, a peripheral blood lymphocyte or a lymphocyte which has been cultured in the presence of an antigen peptide and IL-2 is limiting-diluted, and cultured in the presence of IL-2 and a feeder cell, a proliferated well is stimulated with a vaccine or a candidate thereof, and the presence or absence of IFN-7 production is measured by ELISA or the like. Herein, efficacy of a vaccine can be assessed by calculating the frequency of a CTL precursor cell in a positive well according to Poisson analysis. Herein, the number of positive cells is the number of antigen-specific CTLs, and as the number is larger, efficacy as a vaccine can be said to be higher.

As used herein, the “adjuvant” is a substance which increases an immune response, or otherwise changes an immune response when mixed with an administered immunogen. The adjuvant is classified into, for example, a mineral, a bacterium, a plant, a synthesis product or a host product, depending on the case.

As used herein, the “pathogen” refers to an organism or a factor which can generate a disease or a disorder in a host.

As used herein, “prophylaxis or prevention” refers to treatment intended not to, concerning a certain disease or disorder, cause such a state before such a state is caused.

As used herein, “therapy” refers to, concerning a certain disease or a disorder, in the case of occurrence of such a state, prevention of exacerbation of the disease or the disorder, preferably to keep the status quo, more preferably mitigation, further preferably dissipation.

Such a therapeutic activity or prevention activity, when determined concerning the vaccine of the present invention, is preferably tested in vitro, then, in vivo before use in a human. For example, examples of the in vitro assay for demonstrating therapeutic usefulness or preventive usefulness of the vaccine of the present invention include the effect of specific binding of the vaccine to a cell strain or a patient tissue sample. Such a test can be determined by utilizing a technique known to a person skilled in the art (e.g. immunological assay such as ELISA). Examples of the in vivo test include a method of testing whether the vaccine has the ability of inducing a neutralizing antibody or not, but are not limited thereto.

As used herein, the “subject” refers to an organism to which the treatment of the present invention is applied, and is also referred to as a patient. Preferably, the patient or the subject can be a human.

The present invention provides a method of treatment, inhibition and prevention by administration of an effective amount of the vaccine of the present invention to a subject. In a preferable aspect, the vaccine of the present invention can be substantially purified (examples include a state where a substance limiting the effect or generating an undesirable side effect is substantially not present).

As used herein, “administering” means giving the vaccine of the present invention or the like, or a pharmaceutical composition containing the same, alone or in combination with other therapeutics, to a host for which treatment is intended. A combination can be administered, for example, either simultaneously as a mixture, separately but simultaneously or parallel, or sequentially. This includes presentation of simultaneous administration of combined drugs or the like as a therapeutic mixture, and includes a procedure of administering combined drugs or the like separately but simultaneously (e.g. a case via separate mucous membranes to the same individual). “Combined” administration further includes separate administration of one of first given, and subsequently secondarily given compounds or drugs.

Administration of the vaccine in the present invention may be performed using any procedure and, preferably, it is advantageous to use a needleless syringe, because administration can be conducted without giving any excessive burden to a patient.

Herein, the needleless syringe in the present invention means a medical instrument for injecting a drug liquid to a skin by moving a piston with a gas pressure or elasticity of an elastic member and administering an ingredient such as a drug subcutaneously, more preferably, into subcutaneous cells, without using a syringe needle. Specifically, for example, ShimaJET™ (manufactured by Shimadzu Corporation), Medi-Jector Vision™ (manufactured by Elitemedical) PenJet™ (manufactured by PenJet) and the like are commercially available.

Determination of termination of preventive treatment by the method of the present invention can be performed by confirming an elicited antibody by using a commercially available assay or instrument.

The present invention also provides a pharmaceutical package or kit including a container containing the medicament of the present invention. Notification of a form determined by a governmental organization regulating production, use or selling of a medicament or a biological product can be arbitrarily attached to such a container, and this notification represents approval by a governmental organization with respect to production, use or selling, for administration to a human.

### (General Technique Used in the Present Description)

The technique used in the present description, unless specifically indicated otherwise, uses well-known conventional techniques in sugar chain science, microfluidex, microfabrication, organic chemistry, biochemistry, genetic engineering, molecular biology, microbiology, genetics and associated fields, within the technical scope of the art. Such a technique is sufficiently explained in the following exemplified references and also in references cited in other places in the present description.

Microfabrication is described, for example, in Campbell, S. A. (1996). The Science and Engineering of Microelectronic Fabrication, Oxford University Press; Zaut, P. V. (1996). Micromicroarray Fabrication: a Practical Guide to Semiconductor Processing, Semiconductor Services; Madou, M. J. (1997). Fundamentals of Microfabrication, CRC1 5 Press; Rai-Choudhury, P. (1997). Handbook of Microlithography, Micromachining, & Microfabrication: Microlithography, and an associated part of them is incorporated into the present description as reference.

A molecular biological procedure, a biochemical procedure, a microbiological procedure, and a sugar chain scientific procedure used in the present description are well-known and conventionally used in the art, and are described in, for example, Maniatis, T. et al. (1989). Molecular Cloning: A Laboratory Manual, Cold Spring Harbor and its 3^{rd} Ed. (2001); Ausubel, F. M., et al. eds, Current Protocols in Molecular Biology, John Wiley & Sons Inc., NY, 10158 (2000); Innis, M. A. (1990). PCR Protocols: A Guide to Methods and Applications, Academic Press; Innis, M. A. et al. (1995). PCR Strategies, Academic Press; Sninsky, J. J. et al. (1999). PCR Applications: Protocols for Functional Genomics, Academic Press; Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues: A Practical Approach, IRL Press; Adams, R. L. et al. (1992). The Biochemistry of the Nucleic acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (1996). Bioconjugate Techniques, Academic Press; Method in Enzymology 230, 242, 247, Academic Press, 1994; and Separate Volume Experimental Medicine “Gene Introduction & Expression Analysis Experimental Method” Yodosha Co., Ltd., 1997, and an associated part (which can be all) of them is incorporated into the present description as reference.

### (Explanation of Preferable Embodiments)

Explanation of preferable embodiments will be described below, but these embodiments are exemplification of the present invention, and it should be understood that the scope of the present invention is not limited to such preferable embodiments. It should be understood that a person skilled in the art can easily perform alteration, change or the like within the scope of the present invention, by referring to the following preferable examples.

### (Epitope)

In one aspect, the present invention provides an epitope specific for HHV-6B, including a sequence of at least 5 consecutive amino acids including at least E, or a sequence in which when E is changed to Q, C at the position 487 and G at the position 489 are conserved, among an amino acid sequence shown at the position 484 to the position 496 of SEQ ID No.: 2 (QALCEGGHVFYNP) or an altered sequence thereof. Preferably, the epitope of the present invention consists of an amino acid sequence shown in the position 484 to the position 496 of SEQ ID No.: 2 (QALCEGGHVFYNP).

In one embodiment, the epitope of the present invention includes at least 6 consecutive amino acids, preferably 7 consecutive amino acids, 8 consecutive amino acids, 9 consecutive amino acids, 10 consecutive amino acids, 11 consecutive amino acids, 12 consecutive amino acids, or 13 consecutive amino acids (the full length of QALCEGGHVFYNP), among the amino acid sequence shown in the position 484 to the position 496 of SEQ ID No.: 2 (QALCEGGHVFYNP) or an altered sequence thereof. Preferably, the epitope includes at least 5 consecutive amino acids, preferably 6 consecutive amino acids, preferably 7 consecutive amino acids, 8 consecutive amino acids, 9 consecutive amino acids, 10 consecutive amino acids, 11 consecutive amino acids, 12 consecutive amino acids, or 13 consecutive amino acids (the full length of QALCEGGHVFYNP), among QALCEGGHVFYNP. Such an epitope can be specified using a method well-known in the art using this information of the present invention.

The epitope of the present invention is not limited to the aforementioned epitopes. That is, it is understood that, for example, using a known technique such as Pepscan and based on information described in the present description, a person skilled in the art can appropriately further make a specific sequence of the neutralizing epitope of the present invention, as described in the following.

In another embodiment, importance of formation of a complex between gQ1 and gQ2 in the neutralizing activity is one of the important characteristics in the present invention. Therefore, it is understood that, based on formation of a complex between gQ1 and gQ2 in the neutralizing activity, a person skilled in the art can appropriately further make a specific sequence of the neutralizing epitope of the present invention

### (Antigen)

In one aspect, the present invention provides an antigen containing the epitope of the present invention. It is understood that an epitope to be contained in the antigen of the present invention can take any embodiment described in (Epitope) in the present description.

In one embodiment, the antigen of the present invention contains amino acids of the position 1 to the position 484 of SEQ ID No.: 2 and the epitope of the present invention. In one specific example, the antigen of the present invention contains amino acids of the position 1 to the position 496 of SEQ ID No.: 2. In one specific example, the antigen of the present invention contains the full length of BgQ1 (SEQ ID No.: 2).

### (Antibody)

In one aspect, the present invention provides an antibody to the epitope of the present invention.

Therefore, in a preferable antibody, or an antigen binding fragment or HHV-6B binding molecule thereof, variable domains of a heavy chain and a light chain are derived from a human and, for example, can have a sequence shown in an altered body of an antibody specifically described in the present description (examples include ones including substitution and insertion, addition or deletion of one or a few amino acids, but are not limited thereto). A constant region domain preferably includes a suitable human constant region domain, for example, a domain described in Kabat E. A. et al., US Department of Health and Human Services, Public Health Service, National Institute of Health. A CDR region can be found out by fitting an amino acid sequence of a variable region to database of an amino acid sequence of an antibody produced by Kabat et al. (“Sequence of Proteins of Immunological Interest” US Dept. Health and Human Services, 1983) to examine homology. Concerning a sequence of a CDR region, an altered body in accordance with at least one addition and insertion, substitution or deletion is also included in the present invention, as far as it is within such a scope that the biological activity (e.g. binding activity or neutralizing activity) desired by the present invention is retained. In addition, a sequence having homology with each CDR region of 90 to 100% is exemplified.

Monoclonal antibodies generated to all proteins seen in a human in nature can be typically produced in a non-human system, for example, in a mouse. As a direct result of this, when administered to a human, a xenogeneic antibody as produced by a hybridoma elicits an undesirable immune response predominantly mediated with a constant part of a xenogeneic immunoglobulin. This obviously limits such use of an antibody that administration over a long term is impossible. For this reason, use of a single chain, a single domain, a chimera, CDR grafting, or particularly, a human antibody which is expected not to exhibit a substantial allergy response when administered to a human is particularly preferable. Preferably, the monoclonal antibody of the present invention includes a light chain including a sequence shown in SEQ ID No.: 10 and a heavy chain including a sequence shown in SEQ ID No.: 12 (preferably, these sequences are full length sequences). In another embodiment, it is understood that the antibody or the antibody binding fragment of the present invention includes one or a plurality of CDRs, and/or one or a plurality of framework regions, among a light chain including a sequence shown in SEQ ID No.: 10 and a heavy chain including a sequence shown in SEQ ID No.: 12. Concerning these frameworks and CDRs, Kabat et al. (1991) Sequences of Proteins of Immunological Interest (5th edition, Public Health Service, National Institute of Health, Bethesda, Md.) and Clothia and Lesk (1987) J. Mol. Biol. 196: 901-917 can be referenced.

As is well-known, by a minor change such as deletion, addition, insertion or substitution of one amino acid or a plurality of amino acids, a protein having substantial identity, which corresponds to the original protein, can be produced.

A constant part of a human heavy chain can be a γ₁, γ₂, γ₃, γ₄, μ, α₁, α₂, δ or ε type, preferably a γ type, more preferably a γ₁ type and, on the other hand, a constant part of a human light chain can be a κ or λ type (including λ₁, λ₂ and λ₃ subtypes), and preferably a κ type. Amino acid sequences of all of these constant parts are provided by Kabat et al.

The antibody of the present invention can be produced using any method well-known in the art. Exemplification of such a method is described in Examples, but is not limited thereto. First, by immunizing an animal using an antigen, an antibody is produced.

Herein, in preparation of an antigen, a peptide of a part of an amino acid sequence of a part of an antigen prepared by a recombinant DNA method or chemical synthesis is exemplified. Such a method is exemplified in Examples. The resulting peptide or the like is mixed with an adjuvant, and is used as an antigen. Examples of the adjuvant include Freund's complete adjuvant, Freund's incomplete adjuvant and the like, and any of them may be mixed.

In addition, concerning a monoclonal antibody, a monoclonal antibody-producing hybridoma can be obtained by collecting a spleen or a lymph node from a mammal, and fusing an antibody-producing cell obtained therefrom with a myeloma cell. A method of cell fusion can be performed by a known method and, for example, the hybridoma can be prepared according to the method of Koehler & Milstein (Nature, 256, 495-497 (1975)). In order to prepare a specific antibody recognizing an objective protein, an objective animal (e.g. mouse) is immunized according to the above-described method. A sufficient rise in a blood antibody titer is confirmed, and blood is collected, or a spleen cell is separated. A hybridoma producing a monoclonal antibody, particularly, a monoclonal antibody recognizing a C-terminal or a ring can be prepared by fusing the thus-separated spleen cell with a myeloma cell. The spleen cell is derived from an animal immunized as described above, preferably, a mouse. The myeloma cell is derived from a mammal, and is preferably a mouse myeloma cell. For fusing a cell, polyethylene glycol or the like can be used. By screening and cloning the hybridoma obtained by fusion, a desirable hybridoma can be selected. For preparing a monoclonal antibody, the resulting hybridoma is cultured in vitro or in vivo. Preferably, the hybridoma is cultured in vivo. For example, in order to produce ascites containing a mouse monoclonal antibody, the hybridoma is administered to a mouse intraperitoneally. A monoclonal antibody can be easily purified from the produced ascites by a method known to a person skilled in the art. It is preferable to collect a spleen cell from an immunized animal on 3 to 10 days after final immunization, but the present invention is not limited thereto.

In order to obtain a hybridoma from the resulting immunized cell, for example, by the method described in “Molecular Cellular Biology Fundamental Experimental Method” (Nankodo, Takekazu Horie et al. 1994) or the like, for the purpose of obtaining a cell which can be sub-cultured, a hybridoma can be obtained by fusing a plasmacytoma cell with an immune cell producing an antibody, for example, in the presence of Sendaivirus and polyethylene glycol. As the plasmacytoma cell used herein, it is desirable to use a plasmacytoma cell derived from a homogeneous homeothermal animal among homeothermal animals and, for example, when the plasmacytoma cell is fused with a spleen cell obtained using a mouse as an animal to be immunized, it is preferable to use a mouse myeloma cell. As the plasmacytoma cell, a known cell can be utilized.

Concerning a hybridoma, a hybridoma producing an objective antibody can be obtained by selecting a hybridoma on a HAT medium (medium with hypoxanthine, aminopterin and thymidine added thereto), and investigating (screening) binding of an antibody which is secreted in the culturing supernatant with an antigen, in the stage where a colony is confirmed.

Examples of the screening method include a variety of methods which are generally used in detecting an antibody, such as a spot method, an aggregation reaction method, a Western blotting method, and an ELISA method. Preferably, for example, as exemplified in Examples, concerning the culturing supernatant of a hybridoma, the screening is carried out according to an ELISA method using reactivity with an objective peptide as an index. By this screening, an objective antibody-producing strain specifically reacting with an antigen such as an objective peptide can be screened.

Cloning of the objective antibody-producing strain obtained as a result of screening can be carried out by a normal limiting dilution method, a soft agar method or the like. The cloned hybridoma can be cultured in a large scale in a serum medium or a serum-free medium, as necessary. According to this culturing, a desired antibody having a relatively high purity can be obtained as the culturing supernatant. Alternatively, by inoculating a hybridoma into an abdominal cavity of a mammal having compatibility with a hybridoma, for example, a mouse, a desired antibody can be recovered in a large amount as mouse ascites. The culturing supernatant of the antibody-producing hybridoma of the present invention and ascites of a mouse or the like can be used as it is as a crude antibody liquid. In addition, these can be purified by ammonium sulfate fractionation, salting out, gel filtration, ion exchange chromatography, an affinity chromatography method or the like, according to a conventional method, to obtain a purified antibody.

A polyclonal antibody is obtained, for example, by collecting blood from a mammal immunized with an immunogen. In the method, as the mammal to be immunized with an immunogen, generally, a rabbit, a goat, a sheep, a mouse, a rat or the like is used.

An immunizing method can be performed, for example, by administering an immunogen to a mammal by intravenous, intradermal, subcutaneous, intraperitoneal injection or the like by a general method. More specifically, for example, an immunogen is diluted with a physiological saline-containing phosphate buffer (PBS), physiological saline or the like to a suitable concentration, and this is used optionally with a normal adjuvant and is administered to a test animal a few times at an interval of 2 to 3 weeks. When a mouse is used, the dose for one time is around 50 to 100 μg per animal. Herein, the adjuvant refers to a substance which potentiates non-specifically an immune reaction to an antigen when administered with an antigen. As the adjuvant which is usually used, a pertussis vaccine, a Freund's adjuvant and the like can be exemplified. On 3 to 10 days after final immunization, by collecting blood of a mammal, anti-serum can be obtained. Anti-serum can be used as it is, or can be purified and used as a polyclonal antibody.

Examples of the method of purifying a polyclonal antibody include a non-specific purifying method and a specific purifying method. The non-specific purifying method is aimed at obtaining mainly an immunoglobulin fraction by a salting out method or an ion exchange chromatography method. Examples of the specific purifying method include an affinity chromatography method with an immobilized antigen.

As used herein, the “immunogen” used when an antibody is prepared, when used in the present description, represents a substance generating an immune response or having the ability to cause an immune response in an organism. The immunogen used in preparing the antibody of the present invention can be prepared using an activated hapten and a carrier protein by an active ester method described in Antibodies: A Laboratory Manual, (1989) (Cold Spring Harbor Laboratory Press) or the like. Alternatively, the immunogen can also be prepared by other methods described in Antibodies: A Laboratory Manual, (1989) (Cold Spring Harbor Laboratory Press) or the like, for example, a carbodiimide method, a glutaraldehyde method or a diazo method.

As used herein, as the “carrier protein” used in preparing an antibody, any of various proteins which are known to enhance antigenicity can be used. Examples thereof include a synthetic polypeptide, in addition to polymer substances such as bovine serum albumin (BSA), bovine thyroglobulin (BTG), and keyhole limpet hemocyanin (KLH).

As used herein, the “hapten” used when an antibody is prepared is a partial or incomplete antigen. The hapten is mainly a substance having a low molecular weight, and it alone does not have the ability to stimulate production of an antibody, but when the hapten is bound with a carrier protein by a chemical method or with a crosslinking agent and immunization is performed as an artificial antigen, an antibody to the hapten can be obtained.

An immunological measuring method can be carried out using the antibody of the present invention. As a single specific antibody used in such an immunological measuring method, a monoclonal antibody which can be stably supplied is desirable, but the antibody is not limited thereto and any molecule can be used. Hereinafter, the method is exemplified using a monoclonal antibody. A sandwich immunological measuring method including a step of immobilizing an antibody (first monoclonal antibody) on a solid phase and incubating this with a sample containing an antigen, a step of further adding a labeled second monoclonal antibody and incubating the resulting mixture, and a step of detecting a generated labeled antigen antibody complex in the mixture is exemplified. Alternatively, in the immunological measuring method of the present invention, a sample, a solid-phased first monoclonal antibody and a labeled second monoclonal antibody may be incubated simultaneously. As the sandwich immunological measuring method, depending on its detecting method, all sandwich immune measuring methods such as a sandwich radioimmunoassay method (RIA method), a sandwich enzyme linked immunosorbent assay method (ETA method), a sandwich fluoroimmunoassay method (FIA method), a sandwich light emission immunoassay method (CLIA method), a sandwich light emission enzyme linked immunosorbent assay method (CLEIA method), and an immunochromatography method based on a sandwich method can be applied. For quantitation, the RIA method and the EIA method are preferable. In the present description, “cross reactivity” refers to immune cross reactivity. When an antibody obtained by immunization with a certain antigen also exhibits a binding reaction with another antigen (associated antigen), this reaction is referred to as a cross reaction. When an amount of a reaction between an objective antigen and an antibody thereof is used as a standard, an extent of an amount of a reaction between an associated antigen and an antibody thereof can be expressed as cross reactivity. In the present description, representatively, when expressed as a relative value (%) of affinity such as 1%, 2%, 3%, 0.5%, 0.2%, 0.1% or the like, it can be said that cross reactivity is low. As the value is lower, cross reactivity is lower, and it is shown that specificity for an objective antigen is possessed. Mainly, due to very similar structure between an objective antigen and an associated antigen, the cross reaction occurs in many cases.

An anti-HHV-6B antibody of the present invention, or an antigen binding fragment or HHV-6B binding molecule thereof can be solid-phased on a carrier such as a microtiter plate, a bead, a tube, a membrane, a filter paper, or a plastic cup and, particularly, a polyethylene bead is suitably used. A sample to be measured can be a sample containing HHV-6B, such as plasma, serum, blood, or urine of a human. The antibody of the present invention, or an antigen binding fragment or HHV-6B binding molecule thereof can be labeled with a radioisotope, an enzyme, a fluorescent substance, a light emitting substance, or in a simple measuring method capable of visual determination, a gold colloid or a coloring latex. The radioisotope used in labeling is ¹⁴C, ³H, ³²P, ¹²⁵I, ¹³¹I or the like and, particularly, ¹²⁵I is suitably used. These can be bound to a monoclonal antibody by a chloramine T method, a peroxidase method, an Iodogen method, or a Vault Hunter method. The enzyme which can be used in labeling includes β galactosidase (βGAL), alkaline phosphatase (ALP), and horseradish peroxidase (HRP). These can be bound to a monoclonal antibody by a periodic acid crosslinking method (Nakane method) or a method of Ishikawa et al. (IGAKU-SHOIN Ltd.; Enzyme Immunosorbent Assay, 3rd edition, 75-127, (1987)). As the fluorescent substance used in labeling, there are fluorescein, fluorescamine, fluorescein isothiocyanate, and tetramethylrhodamine isothiocyanate. As the light emitting substance used in labeling, luciferin, a luminol derivative, and an acridinium ester can be mentioned. In a simple measuring method or the like, a gold colloid and a coloring latex may be used.

According to a preferable embodiment, a sandwich RIA method can be performed. In the sandwich RIA method, specifically, a bead on which a first monoclonal antibody is solid-phased is added to a standard solution or a sample, the mixture is kneaded, and this is incubated at 4° C. to 45° C., preferably 25° C. to 37° C., for 1 to 4 hours, preferably 2 hours (first reaction). After washing, for example, a solution containing a second monoclonal antibody labeled with ¹²⁵I is added, the mixture is incubated at 4° C. to 45° C., preferably 25° C. to 37° C., for 1 to 4 hours, preferably 2 hours to form an antibody/antibody complex on the bead (second reaction). After washing, the amount can be measured by detecting radioactivity of an antigen antibody complex bound to a bead with a gamma counter or the like. According to another preferable embodiment, a sandwich EIA method may be carried out. In the sandwich EIA method, specifically, a bead on which a first monoclonal antibody is immobilized is added to a standard solution or a sample, the mixture is kneaded, and this is incubated at 4° C. to 45° C., preferably 25° C. to 37° C., for 1 to 4 hours, preferably 2 hours (first reaction). After washing, a solution containing a second monoclonal antibody labeled with an enzyme label, for example, horseradish peroxidase (HRP), the mixture is incubated at 4° C. to 45° C., preferably 25° C. to 37° C., for 1 to 4 hours, preferably 2 hours, to form an immune complex consisting of the first antibody and the second antibody on the bead (second reaction). The enzyme activity on a bead is measured by a colorimetric method via a substrate specific for an enzyme, for example, when the labeling enzyme is HRP, tetramethylbenzidine (TMB), thereby, a captured amount on a bead can be measured. Colorimetric quantitation can be performed with a normal spectrophotometer or the like.

The antigen binding ability can be measured as follows: In the Cell ELISA plate for measuring antigen binding, a sample is prepared as follows. Appropriate cells are seeded into 60 wells of a 96-well plate for cell culturing to a cell number of 1×10⁶ cells. This is cultured in a CO₂ incubator for 1 day (RPMI1640 medium containing 10% bovine fatal serum (GIBCO)), to adhere cells. The culturing solution is discarded, and each well is washed with 300 μl of PBS two times. 100 μl of PBS containing 4% paraformaldehyde (hereinafter, also referred to as PFA/PBS) is added to each well, and this is allowed to stand on ice for 10 minutes to solid-phase cells. PFA/PBS is discarded, each well is washed with 300 μl of PBS two times, and this is blocked with 250 μl of DB. 100 μl of an antibody is added to each well, this is incubated at room temperature for 2 hours, and washed with RB, and 100 μl of an alkaline phosphatase-bound second antibody which has been diluted 1000-fold with DB is added. After incubation at room temperature for 1 hour and washing with RB, a substrate solution is added and, then, an absorbance at 405/655 nm is measured with a microplate reader (Bio-Rad).

In one embodiment, the antibody of the present invention is a neutralizing antibody. The neutralizing activity can be measured using the antibody-dependent cytotoxicity as an index. The antibody-dependent cytotoxicity can be measured as follows. That is, the antibody-dependent cytotoxicity by a chromium freeing test can be analyzed. A human peripheral mononuclear cell (PBMC) is separated from peripheral blood of a healthy person using Ficoll-paque PLUS (manufactured by GE Healthcare) according to the package insert. To the separated PBMC, DMEM containing 10% FCS is added to 4×10⁶ cells/ml.

To DMEM containing a suitable number (e.g. 1×10⁶) of appropriate cells, physiological saline containing ⁵¹Cr (manufactured by Perkin Elmer) is added to perform a reaction at 37° C. for 1 hour. Thereafter, the reaction is appropriately washed with DMEM, and DMEM is added to a defined amount (e.g. 5×10⁴/ml). To this cell, the antibody of the present invention or a control antibody (e.g. mouse IgG2a; manufactured by SIGMA-ALDRICH) is added, for example, to react them at 37° C. for 1 hour, and this is added to a 9-well v-bottom plate to an appropriate amount (e.g. 100 μl/well). Thereafter, an appropriate amount, for example, 1004 of PBMC is added to react them at 37° C. for 2 hours. Thereafter, the plate is centrifuged at 500×g and room temperature for 5 minutes, and γ-ray of 100 μl of the supernatant is measured with a measurement equipment (e.g. ARC-7001 (manufactured by Aloka)). The antibody specific cytotoxicity (%) is obtained using the following calculation equation.

Cytotoxicity(%)=(experimental value−natural freeing)/(maximum freeing−natural freeing)×100

According to the common technical knowledge in the art, a person skilled in the art can make a humanized antibody, for example, by the CDR grafting method (e.g. European Patent No. 239400).

The antibody of the present invention can be prepared as a chimeric antibody, and an expression vector of such a chimeric antibody is expressed by connecting a DNA encoding a mouse V region to a DNA encoding a human antibody constant region if a DNA fragment encoding a H chain V region is cloned, thereby, a chimeric anti-human antibody is obtained. A fundamental method of preparing the chimeric antibody includes connecting a leader sequence and a V region sequence present in a cloned cDNA to a sequence encoding a human antibody C region already present in an expression vector of a mammal cell. Alternatively, the method includes connecting a mouse leader sequence and a V region sequence present in a cloned cDNA to a sequence encoding a human antibody C region and, thereafter, connecting this to a mammal cell expression vector. A fragment of a human antibody C region can be a H chain C region of an arbitrary human antibody and a L chain C region of a human antibody and, for example, concerning a human H chain, examples include Cγ1, Cγ2, Cγ3 or Cγ4, and concerning a L chain, examples include Cλ or Cκ, respectively.

In one embodiment, the antibody of the present invention is a monoclonal antibody. In one embodiment, a monoclonal antibody described in the present description is MAb KH-1.

The antibody of the present invention reacts with HHV-6B and has no cross reactivity with HHV-6A.

### (Composition and Medicament)

In one aspect, the present invention provides a composition containing the antigen of the present invention. It is understood that as the antigen contained in the composition of the present invention, any embodiment described in items of (Epitope) and (Antigen) in the present description can be used.

In one embodiment, this composition can be a composition for generating a neutralizing antibody of a HHV-6B virus.

In a preferable embodiment, the antigen used in the present invention is HHV-6B gQ1. Without wishing to be bound by any theory, this antigen is used since it has been confirmed that the neutralizing activity is remarkably stimulated by using the full length.

In one embodiment, the composition of the present invention further contains HHV-6B gQ2. Without wishing to be bound by any theory, it is preferable to add gQ2 because it has been found out that the recognition grows stronger when HHV-6A gQ1 is co-expressed with HHV-6B gQ2, although HHV-6B gQ1 is recognized without addition of gQ2. That is, it is thought that, by interaction between gQ1 and gQ2, the neutralizing antibody prepared in the present invention recognizes a steric structure of the formed gQ1. The steric structure formed by binding of gQ1 and gQ2 is useful in the point that the structure serves as a target of HHV-6B infection neutralization and identification of a molecule which inhibits this binding can lead to development of a therapeutic.

In one preferable embodiment, HHV-6B gQ1 and HHV-6B gQ2 contained in the composition of the present invention have formed a complex. Without wishing to be bound by any theory, this is because it was found out in the present invention that there is a high possibility that the formation of a complex of gQ1 and gQ2 is important in a target of infection neutralization. Without wishing to be bound by any theory, HHV-6 enters a cell, probably, by an intracellular route. Envelope proteins gH/gL/gQ1/gQ2 (gH/gL/gO) and gB function in a process of virus adhesion and penetration. This is because HHV-6A utilizes human CD46 as a cell receptor, but HHV-6B seems unlikely to do so.

In one embodiment, HHV-6B gQ1 and HHV-6B gQ2 contained in the composition of the present invention are co-expressed in a cell. Without wishing to be bound by any theory, this is because it is thought that co-expression is preferable for forming a complex because the recognition grows stronger when HHV-6A gQ1 is co-expressed with HHV-6B gQ2 although the monoclonal antibody prepared in the present invention recognizes HHV-6B gQ1 without co-expression of gQ2. The composition of the present invention can be a medicament.

In another aspect, the present invention provides a medicament containing the antigen of the present invention. It is understood that as the antigen contained in the medicament of the present invention, any embodiment described in items regarding the composition among (Epitope), (Antigen) and (Composition and medicament) in the present description can be used. The compound of the present invention or a pharmaceutically acceptable salt thereof can be administered alone, but it is usually preferable to provide it as various medical preparations. In addition, such medical preparations are used in animals and humans.

### (Demonstration of Therapeutic Activity or Preventive Activity)

The compound or the pharmaceutical composition of the present invention is tested for the desired therapeutic activity or preventive activity, preferably, in vitro before use in a human and, then, in vivo. Examples of an in vitro assay for demonstrating therapeutic usefulness or preventive usefulness of the compound or the pharmaceutical composition include the effect of the compound on a cell strain or a patient tissue sample. The effect of the compound or the composition on a cell strain and/or a tissue sample can be determined by utilizing a technique known to a person skilled in the art (examples include a cell lysis assay, but are not limited thereto). Examples of the in vitro assay used for determining whether administration of a particular compound is shown or not, according to the present invention, include an in vitro cell culturing assay. In this assay, a patient tissue sample is proliferated in the culture, and is exposed to the compound, or otherwise the compound is administered, and the effect of the compound on a tissue sample is observed.

The present invention provides a method of treatment, inhibition and prevention by administering an effective amount of an ingredient such as a vaccine or a composition to a subject. In a preferable aspect, an ingredient of the present invention can be an ingredient which has been substantially purified (examples include a state where a substance limiting the effect or generating an undesirable side effect is not substantially present). Examples of the subject preferably include animals such as a cow, a pig, a horse, a chicken, a cat and a dog, but are not limited thereto, and the subject is preferably a mammal, and most preferably a human.

When the present invention is used as a medicament, the medicament of the present invention can further contain a pharmaceutically acceptable carrier. Examples of the pharmaceutically acceptable carrier contained in the medicament of the present invention include any substances known in the art.

It is preferable that, as an administration route of the composition, the medicament, the vaccine or the like of the present invention, an administration route which is most effective upon therapy is used, and examples include an oral route and parenteral routes such as rectal, intraoral, subcutaneous, intramuscular, and intravenous routes. As a dosage form, there are capsules, tablets, granules, powders, syrups, emulsions, suppositories, injectables and the like. A liquid preparation such as an emulsion or a syrup which is suitable for oral administration can be produced using water, saccharides such as sucrose, sorbit, and fructose, glycols such as polyethylene glycol and propylene glycol, oils such as a sesame oil, an olive oil and a soybean oil, antiseptics such as p-hydroxybenzoic acid esters, flavors such as strawberry flavor and peppermint. In addition, capsules, tablets, powders, granules and the like can be produced using excipients such as lactose, glucose, sucrose, and mannit, disintegrating agents such as starch and sodium alginate, lubricants such as magnesium stearate and talc, binders such as polyvinyl alcohol, hydroxypropylcellulose and gelatin, surfactants such as fatty acid esters, and plasticizers such as glycerin.

Examples of such a suitable formulation material or pharmaceutically acceptable carrier include antioxidants, preservatives, coloring materials, flavor materials, and diluents, emulsifiers, suspending agents, solvents, fillers, bulking agents, buffers, delivery vehicles, diluents, excipients and/or pharmaceutical adjuvants, but are not limited thereto. Representatively, the medicament of the present invention is administered in a form of a composition containing an isolated pluripotent stem cell, or an altered body or a derivative thereof together with one or more physiologically acceptable carriers, excipients or diluents. For example, a suitable vehicle can be water for injection, a physiological solution, or an artificial cerebrospinal fluid, and other substances can be generally supplemented to a composition for parenteral delivery.

An acceptable carrier, excipient or stabilizer used in the present description is non-toxic to a recipient, and preferably is inactive in a medication amount and a concentration used. Preferable examples thereof include a phosphate salt, a citrate salt, or other organic acids; ascorbic acid, α-tocopherol; low-molecular polypeptides; proteins (e.g. serum albumin, gelatin and immunoglobulin); hydrophilic polymers (e.g. polyvinylpyrrolidone); amino acids (e.g. glycine, glutamine, asparagine, arginine, and lysine); monosaccharide, disaccharide and other carbohydrates (including glucose, mannose, and dextrin); chelating agents (e.g. EDTA); sugar alcohols (e.g. mannitol and sorbitol); salt forming counter ions (e.g. sodium); as well as/or nonionic surface activating agents (e.g. Tween, pluronic and polyethylene glycol (PEG)), but are not limited thereto.

Examples of the suitable carrier include neutral buffered physiological saline, or physiological saline mixed with serum albumin, Preferably, a product thereof is formulated as a lyophilizing agent using a suitable excipient (e.g. sucrose). Other standard carriers, diluents and excipients can be optionally contained. Other illustrative compositions include a Tris buffer having a pH of 7.0 to 8.5 and an acetate buffer having a pH of 4.0 to 5.5, and these may further include sorbitol or a suitable substitute thereof.

A preparation suitable for parenteral administration consists of a sterilized aqueous preparation containing an active compound, preferably isotonic with blood of a recipient. For example, in the case of an injection, a solution for injection is prepared using a carrier consisting of a salt solution, a glucose solution or a mixture of brine and a glucose solution, or the like.

A local preparation is prepared by dissolving or suspending an active compound in one or more kinds of media, for example, a mineral oil, petroleum, a polyhydric alcohol or other bases used in a local medical preparation. A preparation for intestinal administration is prepared using a normal carrier, for example, cacao butter, a hydrogenated fat, a hydrogenated fatty carboxylic acid or the like, and is provided as a suppository.

In the present invention, also in a parenteral agent, one or more kinds of auxiliary ingredients selected from glycols, oils, flavors, antiseptics (including antioxidants), excipients, disintegrating agents, lubricants, binders, surfactants, and plasticizers exemplified in an oral agent may be added.

The medicament, the vaccine or the like of the present invention can be administered orally or parenterally. Alternatively, the medicament or the like of the present invention can be administered intravenously or subcutaneously. When systemically administered, the medicament or the like used in the present invention can be in the form of a pharmaceutically acceptable aqueous solution, containing no pyrogen. Preparation of such a pharmaceutically acceptable composition can be easily performed by a person skilled in the art in view of the pH, isotonicity, stability and the like. In the present description, an administration method can be oral administration, parenteral administration (e.g. intravenous administration, intramuscular administration, subcutaneous administration, intradermal administration, mucosal administration, rectal administration, intravaginal administration, local administration to an affected part, dermal administration etc.). A formulation for such administration can be provided in any preparation form. Examples of such a preparation form include solutions, injectables, and sustained-release agents.

The medicament or the like of the present invention can be prepared and preserved in a form of a lyophilized cake or an aqueous solution, by mixing with a physiologically acceptable carrier, excipient or stabilizer (see Japanese Pharmacopoeia 16th edition, Supplement thereof or Advanced edition thereof, Remington's Pharmaceutical Sciences, 18th Edition, A. R. Gennaro, ed., Mack Publishing Company, 1990 etc.), and a sugar chain composition having a desired degree of purity, if necessary.

An amount of the sugar chain composition used in the treatment method of the present invention can be easily determined by a person skilled in the art in view of a use purpose, a subject disease (kind, severity etc.), age, weight, sex, and health history of a patient, form or kind of a cell and the like. The frequency of application of the treating method of the present invention to a subject (or a patient) can also be easily determined by a person skilled in the art in view of a use purpose, a subject disease (kind, severity etc.), age, weight, sex, and health history of a patient, and therapeutic process. Examples of the frequency include administration of every day to once per a few months (e.g. once per one week to once per one month). It is preferable that administration of once per one week to one month is applied while following the course.

The effective dose and the number of times of administration of the compound of the present invention or a pharmaceutically acceptable salt thereof is different depending on the dosage form, age or weight of a patient, nature or severity of the symptom to be treated or the like, but usually, the dose is 0.01 to 1000 μg/person, preferably 5 to 500 μg/person per one day, and it is preferable that the number of times of administration is once a day, or the compound is administered by division.

In an aspect, the present invention provides a vaccine containing the antigen of the present invention. It is understood that as the antigen contained in the vaccine of the present invention, any embodiment described in items concerning the composition and the medicament among (Epitope), (Antigen) and (Composition and medicament) in the present description can be used.

In the present description, the immunological effect of the vaccine can be confirmed using any method known in the art. Examples of such a method include CTL precursor cell frequency analysis, an ELISPOT method, a tetramer method, and a real time PCR method, but are not limited thereto. As an illustrative explanation, in the CTL precursor cell frequency analysis, a peripheral blood lymphocyte or a lymphocyte cultured in the presence of an antigen peptide and IL-2 is limiting-diluted, cultured in the presence of IL-2 and a feeder cell, a proliferated well is stimulated with a vaccine or a candidate thereof, and the presence or absence of IFN-γ production is measured by ELISA or the like. Herein, in a positive well, efficacy of a vaccine can be assessed by calculating the frequency of CTL precursor cells according to Poisson analysis. Herein, the number of positive cells is the number of antigen-specific CTLs, and as the number is larger, efficacy as a vaccine can be said to be higher.

The vaccine of the present invention may be prepared with an adjuvant. Regarding the adjuvant, adjuvants known in the art can be utilized, and alum or the like can be utilized.

The vaccine of the present invention can be utilized in prevention or therapy or both of them of a disease caused by HHV-6B (e.g. exanthema subitum).

### (Screening)

In one aspect, the present invention provides a method of screening an inhibitor of a HHV-6B virus. This method includes A) a step of providing HHV-6B gQ1 and HHV-6B gQ2; B) a step of contacting a test substance with the HHV-6B gQ1 and the HHV-6B gQ2 under the condition in which the HHV-6B gQ1 and the HHV-6B gQ2 are bound; and C) a step of observing binding between the HHV-6B gQ1 and the HHV-6B gQ2, wherein when the binding is inhibited, it is determined that the test substance is an inhibitor of a HHV-6B virus.

In implementation of the present invention, HHV-6B gQ1 and HHV-6B gQ2 can be provided by any method in the art. For example, those isolated from a natural product may be used, or those obtained by expression based on a recombinant procedure disclosed in the present description, or using a known sequence may be used. Alternatively, those expressed in a cell themselves may be provided.

As the condition under which HHV-6B gQ1 and HHV-6B gQ2 used in the present invention are bound, any condition known in the art may be used, and any condition of immunoprecipitation is typical. For example, the condition described in Examples is exemplified, but the condition described in Examples may be used with appropriate alternation.

Observation of binding between the HHV-6B gQ1 and the HHV-6B gQ2 implemented in the present invention can be carried out using any technique known in the art. As such an observation technique, for example, the observation technique described in Examples (e.g. Western blotting) is exemplified, or the condition described in Examples may be used with appropriate alteration.

In one embodiment, the HHV-6B gQ1 and the HHV-6B gQ2 used in the present invention can be used in a form co-expressed in a cell.

In one embodiment, in the screening method of the present invention, in the step A), further, gL and gH can be provided. Without wishing to be bound by any theory, gL and gH are provided because that the presence of gL and gH in formation of a steric structure is thought to be closer to the natural state and screening mimicking the state of nature can be carried out, but the present invention is not limited to this. It is understood that the screening itself can be carried out without gL and gH.

In another aspect, the present invention provides a method of screening a neutralizing epitope of a HHV-6B virus. This method includes: A) a step of providing an antibody containing an antigen determining region (CDR) in SEQ ID No.: 10 and SEQ ID No.: 12 or an antigen binding fragment thereof; B) a step of contacting a plurality of peptides being a candidate for the antibody or an antigen binding fragment thereof under the condition in which an epitope is bound; and C) a step of determining a sequence having identity or similarity in the plurality of peptides bound to the antibody or an antigen binding fragment thereof, and selecting the sequence having identity or similarity as a neutralizing epitope.

This method can be carried out using any technique known in the art. As such a condition under which an epitope is bound or the technique for contact, for example, those described in Examples are exemplified, or the conditions described in Examples may be used with appropriate alteration. Observation of binding can be carried out using any technique known in the art. As such an observation technique, for example, the observation technique described in Examples (e.g. Western blotting) is exemplified, or the condition described in Examples may be used with appropriate alteration. Determination of a sequence having identity or similarity in a plurality of peptides bound to an antibody or an antigen binding fragment thereof can also be carried out using any technique known in the art (e.g. Pepscan). In the present invention, the antibody containing an antigen determining region (CDR) in SEQ ID No.: 10 and SEQ ID No.: 12 or an antigen binding fragment thereof may contain a framework sequence or a full length sequence of an antibody, if necessary.

### (Kit)

In one aspect, the present invention provides a kit for screening an inhibitor of a HHV-6B virus. This kit includes A) HHV-6B gQ1; B) HHV-6B gQ2; and C) a means for providing the condition under which the HHV-6B gQ1 and the HHV-6B gQ2 are bound, wherein when a test substance is contacted with the HHV-6B gQ1 and the HHV-6B gQ2 under the condition in which the HHV-6B gQ1 and the HHV-6B gQ2 are bound, if the binding is inhibited, it is determined that the test substance is an inhibitor of a HHV-6B virus. It is understood that, in the kit of the present invention, any embodiment described in the item of (Screening) can be utilized.

In one embodiment, the HHV-6B gQ1 and the HHV-6B gQ2 used in the present invention can be provided in a form co-expressed in a cell, in the kit of the present invention.

In one embodiment, the kit of the present invention may further include gL and gH.

In another aspect, the present invention provides a kit for screening a neutralizing epitope of a HHV-6B virus. This kit includes: A) a means for providing an antibody containing an antigen determining region (CDR) in SEQ ID No.: 10 and SEQ ID No.: 12 or an antigen binding fragment thereof; B) a means for contacting a plurality of peptides being a candidate for the antibody or an antigen binding fragment thereof under the condition in which an epitope is bound; and C) a means for determining a sequence having identity or similarity in the plurality of peptides bound to the antibody or an antigen binding fragment thereof, and selecting the sequence having identity or similarity as a neutralizing epitope. It is understood that, in the kit of the present invention, any embodiment described in the item of (Screening) can be utilized. In the present invention, an antibody containing an antigen determining region (CDR) in SEQ ID No.: 10 and SEQ ID No.: 12 or an antigen binding fragment thereof may contain a framework sequence or a full length sequence of an antibody, if necessary.

In any embodiment of the aforementioned aspects, the kit of the present invention may contain an instruction. This instruction is a description to a person carrying out the present invention a screening method of the present invention. This instruction describes wording of instructing a procedure of screening of the present invention. This instruction is produced according to a form defined by supervisory authority of a country where the present invention is carried out, if necessary, and the effect that approval was received from the supervisory authority is explicitly described. The instruction is so-called package insert and is usually provided on a paper medium, but it is not limited thereto, and can be provided in a form of a film adhered to a bottle, or an electronic medium (e.g. a homepage provided on the internet (website) and electronic mail).

Entirety of references such as scientific references, patents and patent applications cited in the present description are incorporated into the present description as reference to the same extent that each is specifically described.

The present invention has been explained by showing a preferable embodiment for easy understanding, as described above. The present invention will be explained below based on Examples, but the aforementioned explanation and following Examples are provided only for the purpose of illustration, and are not provided for the purpose of limiting the present invention. Therefore, the scope of the present invention is not limited to embodiments or Examples specifically described in the present description, and are limited only by the patent claims.

### EXAMPLES

Handling of animals used in the following examples observed a standard defined in Osaka University.

### Example 1

### Preparation of Monoclonal Antibody to HHV-6B

In this example, a monoclonal antibody BgQ202 (HHV-6B gQ1) or KH-1 (anti-HHV-6B) was prepared.

An outline is as follows;

1. A virion is purified from the supernatant of a cell infected with HHV-6B (HST strain).
2. A BALB/c mouse is immunized with a virion inactivated with UV.
3. A hybridoma producing an antibody to a virion constituent factor is prepared.
4. Among them, a plurality of antibodies having the ability to neutralize HHV-6B are separated.

A procedure thereof and the like will be shown.

### (Materials and Methods)

### <Mouse>

A four week old female BALB/c mouse (inbred, Japan SLC, Inc.) was used.

### <Virus>

A HHV-6B virus was purified from the culturing supernatant of a HST strain (K. Takahashi et al., J. Virol., 3161-3163, 1989) using a mononuclear cell (CBMCs). Specifically, the supernatant containing a virion from an infected cell was collected (centrifuged at 2500×g and 4° C. for 15 minutes), and the virus was settled using 20% polyethylene glycol (molecular weight 20 kDa) in the presence of 0.9% NaCl. The precipitate was resuspended, and the suspension was placed on a layer gradient of 5 to 50% Histodenz (Sigma), and centrifuged at 27,000 rpm for 1 hour (Hitachi P40ST-1689 rotor, Hitachi High-Technologies) to separate and purify particles of a HHV-6B virus (Virology, vol. 378, 269, cell and viruses was referenced).

### <Inactivation>

The purified HHV-6B virus was inactivated by UV irradiation. Specifically, inactivation of a virus was performed by exposing the purified HHV-6B virion to UV light using a suitable UV light source.

A virus stock (500 μl) was arranged on a 35 mm tissue culturing dish (IWAKI), and was irradiated with 2,500 J/m² of UV light.

### <Immunization>

The inactivated HHV-6B virion was administered to the BALB/c mouse in a suitable antigen amount by intraperitoneal injection, to immunize the mouse.

### <Preparation of Hybridoma>

A hybridoma producing an antibody to the inactivated HHV-6B virion constituent factor was prepared. The hybridoma was prepared by fusing a myelocytoma and an antibody-producing cell according to a convention method. As the antibody-producing cell, a spleen cell of the immunized BALB/c mouse was used. As the myelocytoma, a myelocytoma of the same kind of a mouse was used.

More specifically, the hybridoma was established by fusing a spleen cell from a hyperimmunized mouse with a non-producing myelocytoma cell strain Sp2/0-Ag14. After selection in a medium containing hypoxanthine/aminopterin, thymidine, a cell secreting a monoclonal antibody (mAb) was screened by an indirect immunofluorescent assay (IFA). A clone secreting an antibody, reactive with an MT cell, infected with HHV-6B (HST strain) and a Sf9 cell infected with baculovirus±REP-(Bac±REP) was expanded, and cloned by a limiting dilution method. Then, ascites having a high antibody titer was accumulated by injecting a hybrid cell cloned into an abdominal cavity of a mouse treated with pristane (Sigma) (J of General Virology, vol. 83, P898, establishment of mAbs was referenced).

As a procedure, specifically, the myelocytoma: the antibody-producing cell, each of which has been mashed, were mixed at an appropriate ratio using a polyethylene tube, and a medium was removed by centrifugation, and the myelocytoma and the antibody-producing cell were fused using polyethylene glycol as a cell fusion promoting substance. Thereafter, this was centrifuged. Then, the supernatant was removed, and cells were cultured in an appropriate medium. The spleen cell concentration was adjusted to an appropriate cell number/ml.

Then, the supernatant of the hybridoma cultured in the medium was dispensed into each well of a plate coated with an antigen (HHV-6B virus). This plate was cultured in a room using a culturing equipment. A half amount of an appropriate medium was suction-removed, and a medium was added. Such an operation was appropriately repeated. The antibody activity was measured by an enzyme antibody method as necessary. In addition, in order to maintain monoclonality, cloning was performed by a limiting dilution method sequentially.

In addition, the supernatant of a hybridoma cultured as described above was separated and purified, and recovered by ion exchange chromatography. In this manner, the supernatant of the hybridoma was purified and used as a monoclonal antibody-producing material.

### <Antibody>

Among the monoclonal antibody-producing materials obtained as described above, an antibody having the ability to neutralize HHV-6B was separated by an appropriate method.

As the neutralizing ability, the neutralizing activity was analyzed based on a known procedure. For example, measurement can be performed using antibody-dependent cytotoxicity as an index. Antibody dependent cytotoxicity can be measured as follows. That is, antibody-dependent cytotoxicity by a chromium freeing test can be analyzed. A human peripheral mononuclear cell (PBMC) is separated from peripheral blood of a healthy person using Ficoll-paque PLUS (manufactured by GE Healthcare) according to the package insert. The separated PBMC is analyzed by adding DMEM containing 10% FCS to 4×10⁶/ml and observing the resultant.

### (Method of Preparing BgQ202A-1)

A monoclonal antibody BgQ202A-1 in which an N-terminal region of HHV-6B (HST strain) gQ1 was expressed as a recombinant protein in Escherichia coli, a BALB/c mouse was immunized with a purified protein, the spleen was collected, the spleen cell and a SP2 cell being a myeloma cell were fused with polyethylene glycol to prepare a hybridoma, thereafter, screening was performed, and the monoclonal antibody was separated as an antibody which was confirmed to specifically recognize HHV-6B gQ1.

In order to obtain the present antibody, preparation of a recombinant protein of an N-terminal region of HHV-6B gQ1 was performed. As a specific method, PCR was performed using BU100-bamF and BU100pstR primers, and employing a cDNA of a HHV-6B HST strain as a template, to amplify the N-terminal region of HHV-6B gQ1. This PCR product was cut with restriction enzymes BamHI and PstI, and cloned into a plasmid pQE30 (QIAGEN) for expressing Escherichia coli, which had been cut with the same restriction enzymes. The present plasmid was introduced into a BL21 strain of Escherichia coli, and a recombinant protein BgQ1-N in which a histidine tag was added to an N-terminal was expressed. BgQ1-N which had been expressed in Escherichia coli in a large amount was purified using a nickel column.

### (Result)

As a result, a monoclonal antibody BgQ202 (HHV-6B gQ1) or KH-1 (anti-HHV-6B) was prepared.

### (Characterization=Sequencing of KH-1)

In order to further characterize KH-1 which is a neutralizing antibody, a gene sequence of this antibody was determined. An amino acid sequence was determined based on a nucleic acid sequence containing a gene sequence encoding an antibody obtained from a hybridoma.

An amino acid sequence thereof is shown below.

Light chain
<SEQ ID No.: 10>
LIRLTIGQAVVSTQSTWGLMRIAVISXGPKFKDKMDFQVQIFSFLLISASVILSRGQIV
LTQSPAIMSASPGEKVTMTCSASSSISYMHWYQQKPGTSPKRWIYDTSKLASGVPARFS
GSGSGTSYSLTISNMESEDAATYYCHQRSRYHTFGGGTRLEIKRADAAPTVSIFPPSSE
QLTSGGASVVCFLNNFYPKDINVKWKIDGS
<X is an arbitrary amino acid>
Heavy chain
<SEQ ID No.: 12>
NTTHYRASSGINAEYMGINICPMSSPQSLKTLTITMGWTWIFILILSVTTGVHSEVQLQ
QSGPELEKPGASVKISCKASGYSFTGYNMNWVKQSNGKRLEWIGNIDPYYGGASYNQKF
KGKATLTVDKSSTTAYMQLQSLTSEDSAVYYCARGGYGRYFDVWGAGTAVTVSSAKTTP
PSVYPLAPGCGDTTGSSVTLGCLVKGYF

In this manner, it was shown that an antibody having the above sequence has the neutralizing activity.

### Example 2

### Determination of Virus Protein Recognized by Monoclonal Antibody to HHV-6B

In this example, a virus protein recognized by a monoclonal antibody to HHV-6B was determined.

### (Materials and Methods)

An MT4 cell infected with HST was collected 12 hours, 24 hours, 48 hours and 72 hours after infection (p.i.). This cell was immobilized together with a primary antibody in cold acetone, and incubated at 37° C. for 1 hour. OHV-2 which is anti-REPmAb recognizes OHV-3 being a nuclear protein which is expressed in an early stage, and recognizes a HHV-6B glycoprotein H (gH) which is expressed in a later stage. After washing for 10 minutes with PBS which is usually used in the art, a goat antibody bound to fluorescein to mouse IgG was added, and to this was added saturated 4′,6′-diamidino-2-phenylindole (DAPI) at a dilution rate of 1:100. This cell was incubated for 20 minutes. After washing as described above, a signal was detected with a confocal microscope (J of General Virology, vol. 83, P848, Immunohistochemical analysis of HST-infected MT9 cells was referenced).

In a HST-infected HMT4 cell, mock and HHV-6B strains were metabolically labeled (³⁵S methionine) for 16 hours, melted, and immunoprecipitated with a monoclonal antibody BgQ202 (HHV-6B gQ1) or KH-1 (anti-HHV-6B) (IP). In both cases, antibodies produced in Example 1 were used.

Immunoprecipitation was resolved by SDS-PAGE (4-12% Tris-glycine gel; Invitrogen). Resolution was performed by fixing, drying and exposing a gel. This result shows that an anti-HHV-6B monoclonal antibody can recognize a glycoprotein Q1.

### (Result)

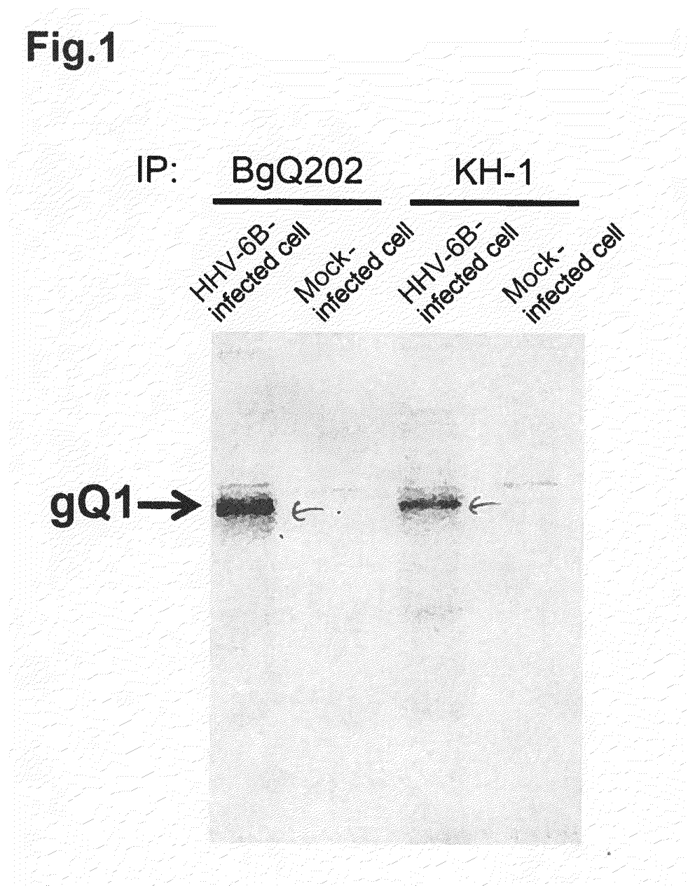

The result is shown in FIG. 1. FIG. 1 shows determination of a virus protein recognized by a monoclonal antibody to HHV-6B. As shown in FIG. 1, an anti-HHV-6B virion monoclonal antibody which can recognize a glycoprotein Q1 was named KH-1.

### Example 3

### Detection of gH/gL/gQ in HHV-6B-Infected Cell by Anti-gQ1 Monoclonal Antibody)

In this example, an experiment for detecting gH/gL/gQ in a HHV-6B-infected cell by an anti-gQ1 monoclonal antibody was performed.

### (Materials and Methods)

A mock or a cell lysate infected with HHV-6B was immunoprecipitated with an anti-gQ1 monoclonal antibody KH-1, and subjected to SDS-PAGE under a reducing condition. A SDS-PAGE gel was electrically transferred to a PVDF membrane, and detection was performed using monoclonal antibodies to gQ1, gH and rgL.

Specifically, a HHV-6-infected cell and a mock-infected cell were dissolved in a radioactive immunoprecipitation assay (RIPA) buffer (0.01 M Tris-HCl [pH 7.4], 0.15 M NaCl, 1% sodium deoxycholate, 1% Nonidet P-40, 0.1% sodium dodecylsulfate [SDS], 1 mM EDTA, 1 mM phenylmethylsulfonyl fluoride). The dissolved protein was separated by SDS-polyacrylamide gel electrophoresis (PAGE), and electrically transferred to a polyvinylidene difluoride (PVDF) membrane for immunoblotting (J of Virology, vol. 78, no. 15, P4610, Immunoblotting, and J of Virology, vol. 78, no. 9, P7972, Preparation of pulse-chase and metabolically labeled proteins and immunoprecipitation experiments were referenced).

### (Result)

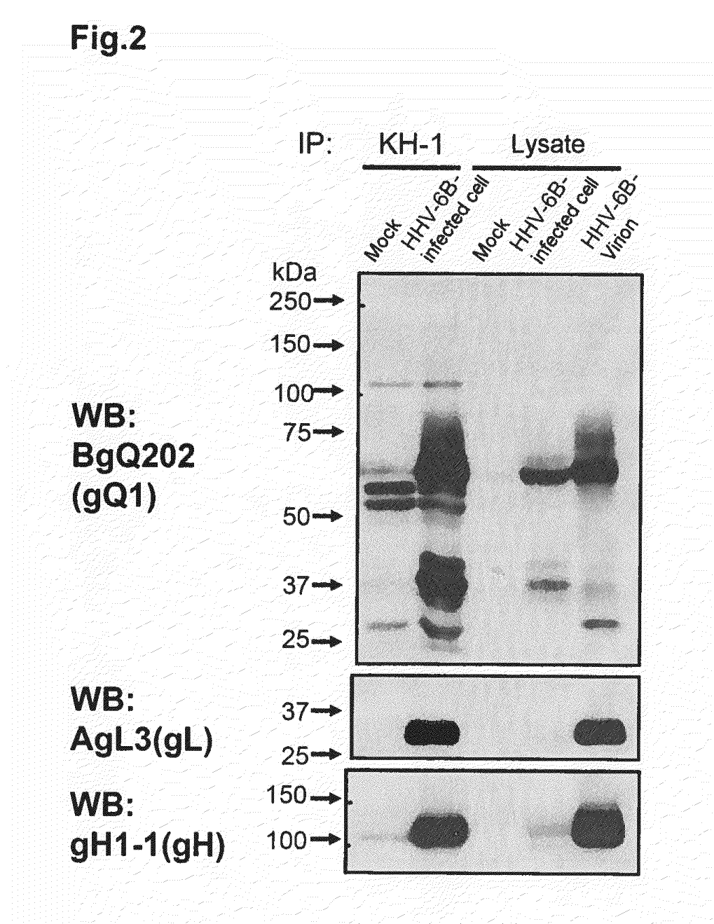

The result is shown in FIG. 2. FIG. 2 shows detection of gH/gL/gQ in a HHV-6B-infected cell by an anti-gQ1 monoclonal antibody. A mock or a cell lysate infected with HHV-6B were immunoprecipitated with an anti-gQ1 monoclonal antibody KH-1, and subjected to SDS-PAGE under a reducing condition. The gel was electrically transferred to a PVDF membrane, and detection was performed using monoclonal antibodies to gQ1, gH and rgL. Since gQ1, gL and gH co-precipitate as a result of reactivity of KH-1, it was shown that KH-1 specifically reacts with HHV-6B gQ1.

In this example, gH/gL/gQ were detected by an anti-gQ1 monoclonal antibody in a HHV-6B-infected cell, and it was found out that these seem to form a complex.

### Example 4

### Confirmation of Neutralizing Activity of Anti-gQ1 Antibody KH-1

In this example, an experiment for confirming the neutralizing activity of an anti-gQ1 antibody KH-1 was performed.

### (Materials and Methods)

For a neutralization assay, a stock of a titered HHV-6B strain HST virus was added to a control antibody or KH-1 at 37° C. for 30 minutes. After incubation, the resulting solution was mixed with a MT4 cell at 37° C. for 1 hour. A virus solution was taken out and washed, cells were incubated in a fresh medium for 12 hours, and stained by an indirect immunofluorescent assay (IFA) using an anti-IE1 rabbit serum.

### (Result)

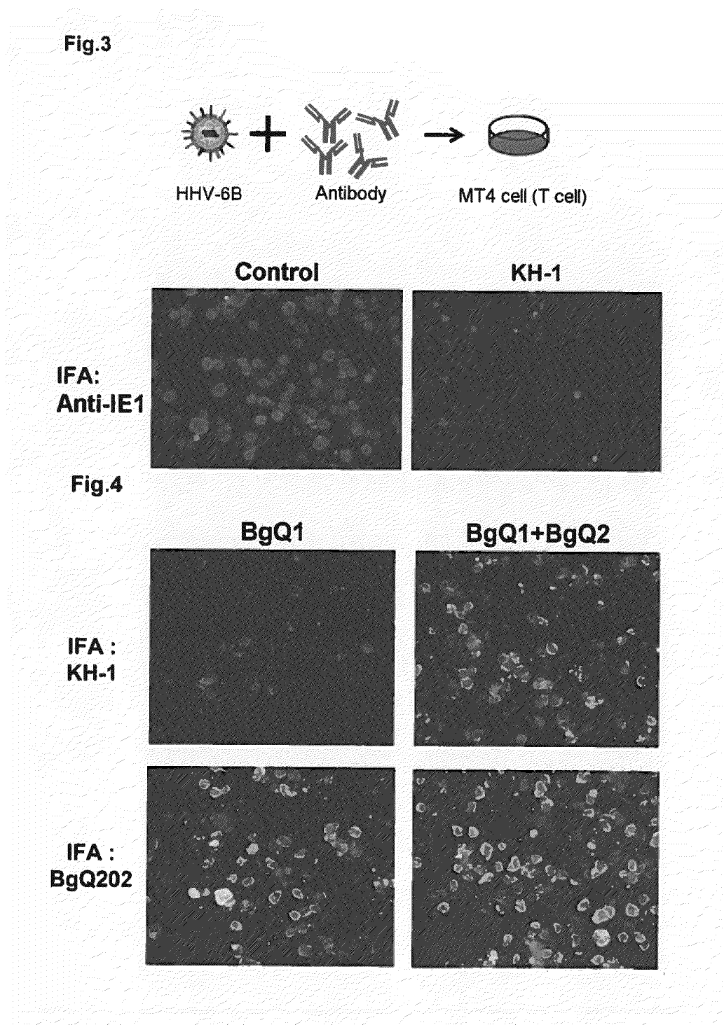

The results are shown in FIG. 3. FIG. 3 is the result showing that KH-1 which is an anti-gal antibody has the neutralizing activity.

### Example 5

### Expression of Protein Recognized by Antibody in gQ1 Transient Expression System

In this example, expression of a protein recognized with an antibody in a gQ1 transient expression system was confirmed.

### (Materials and Methods)

293T cells were co-transfected with a plasmid expressing gQ1 and gQ2. Cells were co-stained using an anti-gQ1 antibody 72 hours after transfection, concerning IFA.

### (Result)

The results are shown in FIG. 4. FIG. 4 shows expression of a protein recognized with an antibody in a gQ1 transient expression system. An expression amount of gQ1 recognized with a KH-1 antibody is increased by addition of gQ2.

### Example 6

### Schematic Diagram of HHV-6B gQ1 Gene Using Various Carboxy Terminal-Detected Mutants and Reaction to Monoclonal Antibody KH-1

In this example, a schematic diagram of a HHV-6B gQ1 gene using various carboxy terminal-detected mutants and their reactivity with a monoclonal antibody KH-1 was investigated.

### (Materials and Methods)

293T cells were co-transfected with a plasmid expressing gQ1 or various gQ1-detected mutants and a plasmid expressing gQ2. The specific procedure was the same as that of Example 5. Cells were transfected using an anti-gQ1 antibody and, after 72 hours, co-stained, concerning IFA. The specific procedure was the same as that of Example 5.

### (Result)

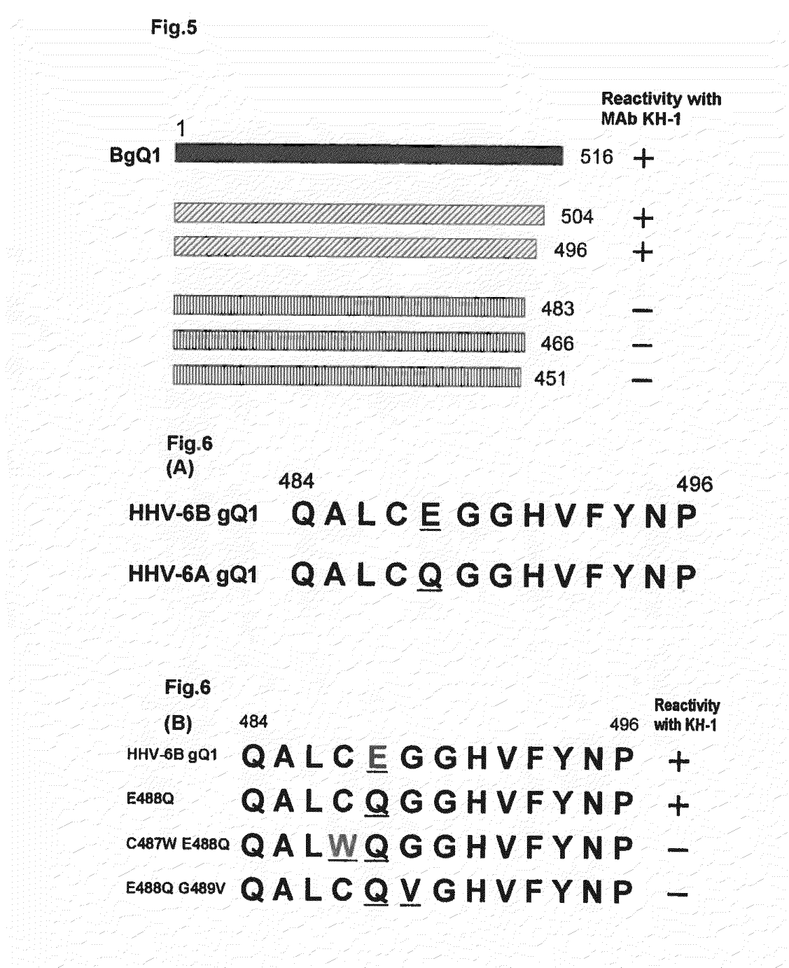

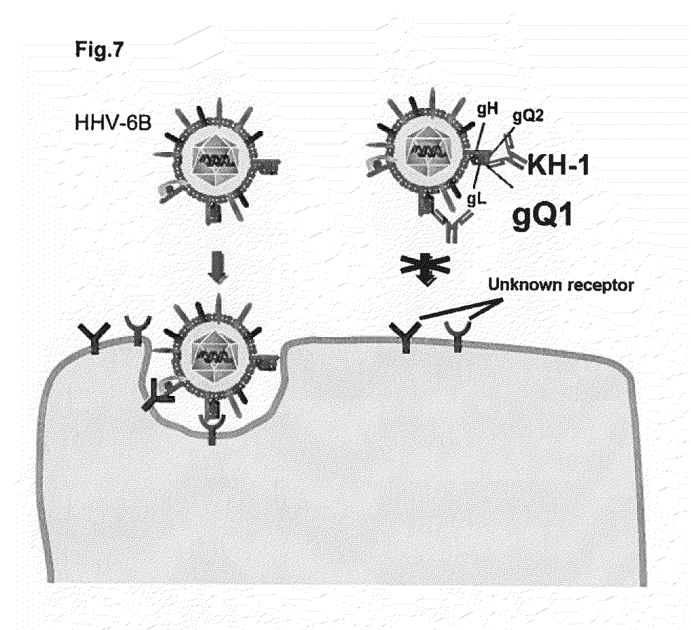

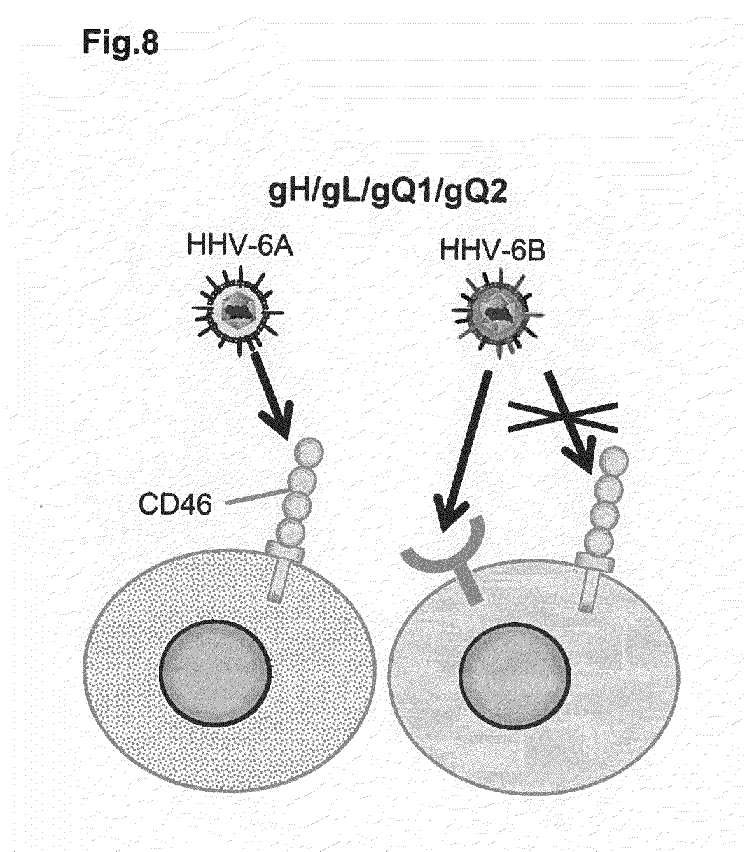

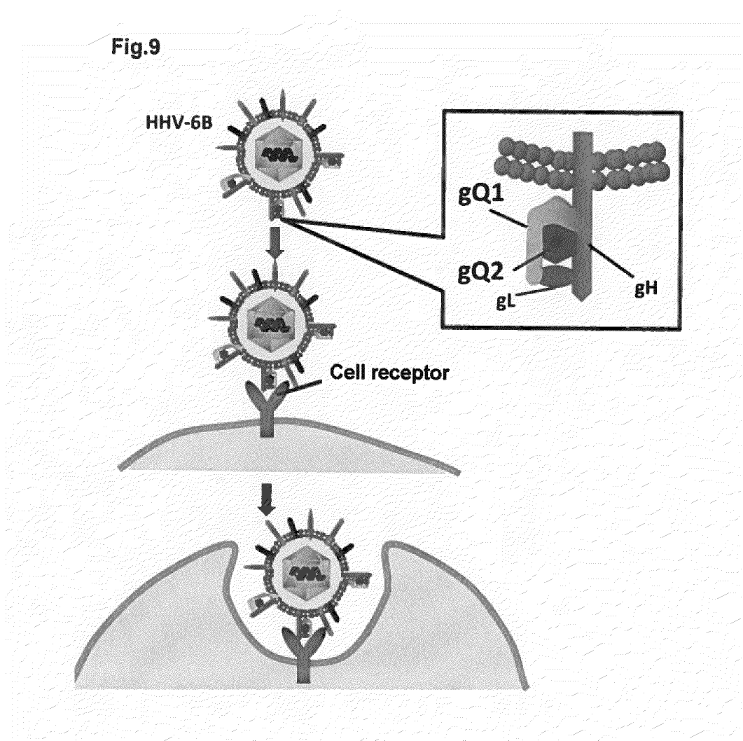

The result is shown in FIG. 5. FIG. 5 shows a schematic diagram of various carboxy terminal-detected mutants of HHV-6B gQ1 gene and their reactivity with a monoclonal antibody KH-1. 293T cells were co-transfected with a plasmid expressing gQ1 or various gQ1-detected mutants and a plasmid expressing gQ2. Cells were co-stained using an anti-gQ1 antibody 72 hours after transfection, concerning IFA. As shown in FIG. 5, mutation at the position 1 to the position 496, the position 1 to the position 504, or the position 1 to the position 516 had reactivity with KH-1. Mutation at the position 1 to the position 483, the position 1 to the position 466, or the position 1 to the position 451 did not have reactivity with KH-1. Therefore, a site recognized by KH-1 is present between the position 484 and the position 496 of gQ1 of HHV-6B.

As shown in FIG. 5, a schematic diagram of various carboxy terminal-detected mutants of HHV-6B gQ1 gene and their reactivity with a monoclonal antibody KH-1 are shown.

### Example 7

### Identification of Neutralization Site of gQ1 of HHV-6A and HHV-6B

In this example, identification of a neutralization site of gQ1 of HHV-6A and HHV-6B was performed.

In this example, the present inventors mapped a neutralization site based on information and the like confirmed in Example 6. FIG. 6A shows alignment between amino acid sequences of gQ1 of HHV-6A and HHV-6B. As apparent from FIG. 6A, it was found out, due to carboxy terminal deletion of gQ1, amino acid residues at the position 484 to the position 496 in a HHV-6B gQ1 sequence are recognized by the monoclonal KM-1. Then, in order to search a HHV-6B-specific amino acid residue in this region, the present inventors compared amino acid sequences of gQ1 of HHV-6A and HHV-68. When sequence comparison was performed, an amino acid position 488 was specifically Glu in HHV-6B. On the other hand, concerning HHV-6A, a corresponding residue was Gln.

In addition, in order to identify a HHV-6B gQ1 epitope site recognized by KH-1, the presence or absence of KH-1 reactivity of a point mutant at the C-terminal was also confirmed. A HHV-6B gQ1 wild type and a point mutant were expressed in 293T cells, and the presence or absence of a reaction thereof was confirmed by IFA using KH-1. The result is shown in FIG. 6B.

As shown in FIG. 6B, reactivity was retained in HHV-6B gQ1 and E488Q, but reactivity disappeared in C487W E488Q and E488Q G489V. Therefore, it was found out that an epitope site of gQ1 recognized by KH-1 is a region containing glutamine at the position 488.

### Example 8

### Vaccine

In this example, a vaccine is produced using the neutralizing antigen of the present invention. This vaccine contains an immunologically defensive amount of an antigen, and can be prepared by a conventional technique.

A vaccine preparation is generally described, for example, in Pharmaceutical Biotechnology, Vol. 61 Vaccine Design—the subunit and adjuvant approach, edited by Power and Newman, Plenurn Press, 1995; New Trends and Developments in Vaccines, edited by Voller et al., University Park Press, Baltimore, Md., U.S.A. 1978. An amount of a protein in each vaccine dosage form is selected as such an amount that an immunologically defensive response is induced without any side effect which is significantly harmful in a typical vaccine. Such an amount can vary depending on what specific immunogen is used. Generally, each dosage form contains 1 to 1000 μg of a protein, preferably 2 to 100 μg, most preferably 4 to 40 μg of a protein, but the content is not limited thereto.

In this example, a protein-bound vaccine can be prepared by conjugating a peptide or a polypeptide (e.g. containing positions 1 to 496 of SEQ ID No.: 2) containing an epitope site determined based on the experiments of Examples 1 to 7 to a protein such as keyhole limpet hemocyanin (KLH) based on a procedure known in the art.

In addition, such a vaccine can be prepared as a vaccine preparation, for example, by combining with a suitable adjuvant such as alum or aluminum hydroxide.

An optimal amount concerning a particular vaccine can be confirmed by a standard test including study of an antibody titer and other responses in a subject. Subsequent to the first inoculation, booster immunization may be given to a subject in, for example, the 4th week. Such an antibody titer of a vaccine can be confirmed by inoculating the vaccine preparation into a mouse or the like and performing a test.

As described above, the present invention has been exemplified using preferable embodiments of the present invention, but it is understood that the scope of the present invention should be construed only by the patent claims. It is understood that the content of patents, patent applications and references cited in the present description should be incorporated into the present description as reference, as if the content thereof itself is specifically described in the present description.

### INDUSTRIAL APPLICABILITY

According to the present invention, an effective vaccine and a useful therapeutic of HHV-6B, as well as a method of screening the same are provided. The present invention finds out applicability in the pharmaceutical industry.

### SEQUENCE LISTING FREE TEXT

SEQ ID No.: 1 is a nucleic acid sequence encoding a full length amino acid sequence of HHV-6B gQ1.

SEQ ID No.: 2 is a full length amino acid sequence of HHV-6B gQ1.

SEQ ID No.: 3 is a nucleic acid sequence encoding a full length amino acid sequence of HHV-6B gQ2.

SEQ ID No.: 4 is a full length amino acid sequence of HHV-6B gQ2.

SEQ ID No.: 5 is a nucleic acid sequence encoding a full length amino acid sequence of HHV-6B gH.

SEQ ID No.: 6 is a full length amino acid sequence of HHV-6B gH.

SEQ ID No.: 7 is a nucleic acid sequence encoding a full length amino acid sequence of HHV-6B gL.

SEQ ID No.: 8 is a full length amino acid sequence of HHV-6B gL.

SEQ ID No.: 9 is a nucleic acid sequence of a light chain of an antibody KH-1.

SEQ ID No.: 10 is an amino acid sequence of a light chain of an antibody KH-1.

SEQ ID No.: 11 is a nucleic acid sequence of a heavy chain of an antibody KH-1.

SEQ ID No.: 12 is an amino acid sequence of a heavy chain of an antibody KH-1.

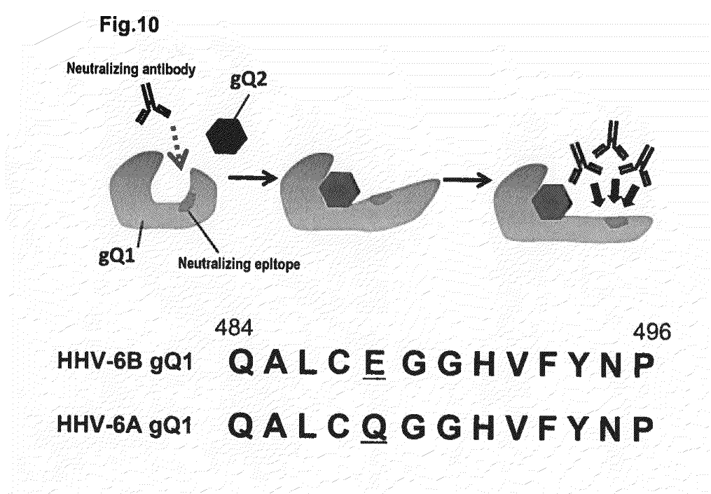

SEQ ID No.: 13 is an amino acid sequence of a human herpesvirus type 6A (HHV-6A) of a part corresponding to the position 484 to the position 496 of SEQ ID No.: 2 (FIG. 6, FIG. 10).

SEQ ID No.: 14 is an amino acid sequence of a HHV-6B E488Q altered sequence of a part corresponding to the position 484 to the position 496 of SEQ ID No.: 2 (FIG. 6)

SEQ ID No.: 15 is a HHV-6B C487W E488Q altered sequence of a part corresponding to the position 484 to the position 496 of SEQ ID No.: 2 (FIG. 6).

SEQ ID No.: 16 is a HHV-6B E488Q G489V altered sequence of a part corresponding to the position 484 to the position 496 of SEQ ID No.: 2 (FIG. 6).

1. An epitope specific for HHV-6B, comprising a sequence of at least 5 consecutive amino acids comprising at least E, or comprising a sequence in which when E is changed to Q, C at the position 487 and G at the position 489 are conserved, among an amino acid sequence shown in the position 484 to the position 496 of SEQ ID No.: 2 (QALCEGGHVFYNP) or an altered sequence thereof.

2. The epitope according to claim 1, comprising an amino acid sequence shown in the position 484 to the position 496 of SEQ ID No.: 2 (QALCEGGHVFYNP).

3. An antibody to the epitope as defined in claim 1 or an antigen binding fragment.

4. The antibody or the antigen binding fragment according to claim 3, having neutralizing activity.

5. The antibody or the antigen binding fragment according to claim 3, which is a monoclonal antibody.

6. The antibody according to claim 5, comprising a light chain comprising a sequence shown in SEQ ID No.: 10 and a heavy chain comprising a sequence shown in SEQ ID No.: 12.

7. An antigen comprising the epitope as defined in claim 1.

8. An antigen comprising the epitope as defined in claim 1, comprising at least the position 1 to the position 496 of amino acids, among SEQ ID No.: 2 (full length of BgQ1).

9. An antigen comprising the epitope as defined in claim 1 comprising a full length BgQ1.

10. A composition comprising the antigen as defined in claim 7.

11. A composition for producing a neutralizing antibody of a HHV-6B virus, comprising the antigen as defined in claim 7.

12. The composition according to claim 11, wherein the antigen is HHV-6B gQ1.

13. The composition according to claim 12, further comprising HHV-6B gQ2.

14. The composition according to claim 13, wherein the HHV-6B gQ1 and the HHV-6B gQ2 have formed a complex.

15. The composition according to claim 13, wherein the HHV-6B gQ1 and the HHV-6B gQ2 are co-expressed in a cell.

16. The composition according to claim 10, which is a medicament.

17. The composition according to claim 10, which is a vaccine.

18. A method of screening an inhibitor of a HHV-6B virus, the method comprising:
A) a step of providing HHV-6B gQ1 and HHV-6B gQ2;
B) a step of contacting a test substance with the HHV-6B gQ1 and the HHV-6B gQ2 under the condition in which the HHV-6B gQ1 and the HHV-6B gQ2 are bound; and
C) a step of observing binding between the HHV-6B gQ1 and the HHV-6B gQ2, wherein when the binding is inhibited, it is determined that the test substance is an inhibitor of a HHV-6B virus.

19. The method according to claim 18, wherein the HHV-6B gQ1 and the HHV-6B gQ2 are co-expressed in a cell.

20. The method according to claim 18, wherein gL and gH are further provided in the step A).

21. A kit for screening an inhibitor of a HHV-6B virus, the kit comprising:
A) HHV-6B gQ1;
B) HHV-6B gQ2; and
C) a means for providing the condition under which the HHV-6B gQ1 and the HHV-6B gQ2 are bound, wherein
in the case where the binding is inhibited when a test substance is contacted with the HHV-6B gQ1 and the HHV-6B gQ2 under the condition in which the HHV-6B gQ1 and the HHV-6B gQ2 are bound, it is determined that the test substance is an inhibitor of a HHV-6B virus.

22. The kit according to claim 21, wherein the HHV-6B gQ1 and the HHV-6B gQ2 are co-expressed in a cell.

23. The kit according to claim 21, further comprising gL and gH.

24. A method of screening a neutralizing epitope of a HHV-6B virus, the method comprising:
A) a step of providing an antibody comprising an antigen determining region (CDR) in SEQ ID No.: 10 and SEQ ID No.: 12 or an antigen binding fragment thereof;
B) a step of contacting a plurality of peptides being a candidate for the antibody or an antigen binding fragment thereof under the condition in which an epitope is bound; and
C) a step of determining a sequence having identity or similarity in the plurality of peptides bound to the antibody or an antigen binding fragment thereof, and selecting the sequence having identity or similarity as a neutralizing epitope.

25. A kit for screening a neutralizing epitope of a HHV-6B virus, the kit comprising:
A) a means for providing an antibody comprising an antigen determining region (CDR) in SEQ ID No.: 10 and SEQ ID No.: 12 or an antigen binding fragment thereof;
B) a means for contacting a plurality of peptides being a candidate for the antibody or an antigen binding fragment thereof under the condition in which an epitope is bound; and
C) a means for determining a sequence having identity or similarity in the plurality of peptides bound to the antibody or an antigen binding fragment thereof, and selecting the sequence having identity or similarity as a neutralizing epitope.
